(19) United States
(12) Patent Application Publication
(10) Pub. No.: US 2020/0106020 A1
(43) Pub. Date: Apr. 2, 2020

(54) BENZO HETEROCYCLIC COMPOUND, DISPLAY PANEL, AND DISPLAY DEVICE CONTAINING THE BENZO HETEROCYCLIC COMPOUND

(71) Applicant: WuHan TianMa Micro-Electronics Co., Ltd., Wuhan (CN)

(72) Inventors: Wei GAO, Wuhan (CN); Lei ZHANG, Wuhan (CN); Qing ZHU, Wuhan (CN); Jinghua NIU, Wuhan (CN); Gaojun HUANG, Wuhan (CN)

(21) Appl. No.: 16/244,158

(22) Filed: Jan. 10, 2019

(30) Foreign Application Priority Data
Sep. 29, 2018 (CN) 201811148058.5

(51) Int. Cl. H01L 51/00; C07D 409/14

(52) U.S. Cl. CPC H01L 51/0061; C07D 409/14; H01L 51/5064; H01L 51/0072; H01L 51/0074; H01L 51/006

(57) ABSTRACT

The present disclosure provides a benzo heterocyclic compound having a structure represented by Formula (I),

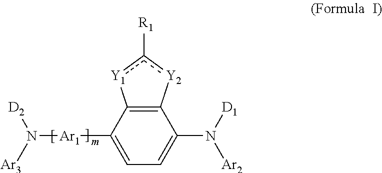

in which, R₁ is selected from a group consisting of hydrogen, C1-C20 alkyl, C1-C20 alkoxy, phenyl, naphthyl, anthracyl, and phenanthryl; Y₁ and Y₂ are each independently selected from a group consisting of carbon, oxygen, and sulfur, and one of Y₁ and Y₂ is carbon; Ar₁ is selected from a group consisting of a substituted or unsubstituted phenyl, naphthyl, anthracyl, phenanthryl, acenaphthylenyl, and an aromatic heterocyclic group; m is 0 or 1; D₁ and D₂ are each independently aryl or heteroaryl, and Ar₂ and Ar₃ are each independently aryl or heteroaryl. When the benzo heterocyclic compound is used as a material of a capping layer (CPL) of an OLED component, high light extraction efficiency can be obtained while color purity reduction of a blue light-emitting component is avoided.

### CROSS-REFERENCE TO RELATED APPLICATIONS

The present application claims priority to Chinese Patent Application No. 201811148058.5, filed on Sep. 29, 2018, the content of which is incorporated herein by reference in its entirety.

### TECHNICAL FIELD

The present disclosure relates to the technical field of organic electroluminescent materials, and particularly, to a benzo heterocyclic compound, a display panel and a display device using the benzo heterocyclic compound.

### BACKGROUND

According to a direction of light emitted from an organic light-emitting layer, organic light-emitting diode (OLED) display devices can be classified into a bottom emission-type OLED display device and a top emission-type OLED display device. In the bottom emission-type OLED display device, light is emitted toward a substrate, a reflection electrode is formed above the organic light-emitting layer, and a transparent electrode is formed below the organic light-emitting layer. If the OLED display device is an active matrix OLED display device, a part of thin film transistors formed therein does not transmit light, resulting in a decrease of light-emitting area. In the top emission-type OLED display device, the transparent electrode is formed above the organic light-emitting layer, and the reflection electrode is formed below the organic light-emitting layer, so that the light is emitted in a direction opposite to the substrate, thereby increasing the light transmission area and brightness.

In order to improve luminescence efficiency, several common methods are known, for example forming folds, photonic crystals, or microlens arrays (MLA) on a surface of the substrate from which light is emitted, coating a covering layer having high refractive index on a transflective electrode having low refractive index, etc. In the case of forming folds or photonic crystals on the surface of the substrate from which light is emitted, an angle distribution of a radiation spectrum of the OLED is affected. In the case of forming a microlens array on the surface of the substrate from which light is emitted, the manufacturing process is complex. On the contrary, the process of coating the covering layer is relatively simple and can significantly improve the luminescence efficiency, and thus gains interest in recent years.

At present, methods for improving performance of the OLED components include: lowering a driving voltage, improving the luminescence efficiency, and prolonging a service life of the devices. In order to continuously improve the performance of the OLED components, it is urgent to innovate not only the structure and manufacturing process of the OLED components, but also electroluminescent materials used in the OLED components.

During a preparation of OLED components by means of vapor deposition, a high-precision metal mask is required to form a covering layer. However, a thermal deformation of the metal mask may lead to a poor positioning accuracy. That is, a melting point of ZnSe is higher than 1100° C. (Appl. Phys. Lett., 2003, 82, 466), and the high-precision mask cannot be accurately deposited onto a specific position. At the same time, inorganic materials are mostly evaporated at high temperature and thus are not suitable for the use of the high-precision mask. An inorganic material film-forming method based on sputtering also causes damage to light-emitting devices, so that the inorganic materials cannot be used to form the covering layer.

In view of a low efficiency in light extraction of OLED components, a capping layer (CPL, also referred as to a cathode covering layer), i.e., a light extraction material, is required to cover a surface of the cathode in the top emission-type device, in order to adjust an optical interference distance, suppress external light emission, and suppress the extinction caused by a movement of surface plasma. Based on principles of optical absorption and refraction, a higher refractive index of the material of such surface covering layer is conducive.

The materials of the cathode covering layer are classified into an inorganic type and an organic type. At present, the materials of CPL are mainly hole transmission-type materials and electron transmission-type materials. An example of the organic light-emitting layer (EL) material known in the prior art is 8-hydroxyquinoline aluminum (hereinafter referred to as Alq3), which is usually used as a green light-emitting material or an electron transmission material. When Alq3 is applied in the cathode covering layer for adjusting the refractive index, it has weak absorption of light having a wavelength near 450 nm emitted by a blue light-emitting device. Therefore, when Alq3 is applied to the blue light-emitting device, a problem of reduced color purity may occur.

The existing CPL materials have following problems: (1) insufficient refractive index and poor light extraction effect; and (2) large difference between the refractive indexes respectively measured in wavelength regions of blue light, green light and red light. Therefore, not all kinds of light can be extracted with a high light extraction efficiency in a light-emitting device that emits blue, green and red light at the same time. In this regards, it is urgent to develop a new CPL material having a refractive index as high as possible at respective wavelengths to improve the extraction efficiency with respect to light having different colors.

### SUMMARY

In a first aspect, the present disclosure provides a benzo heterocyclic compound having a structure represented by Formula (I):

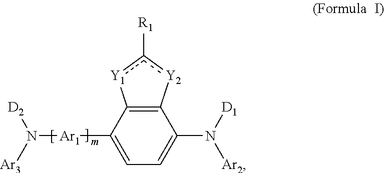

in which wherein R₁ is selected from a group consisting of hydrogen, C1-C20 alkyl, C1-C20 alkoxy, phenyl, naphthyl, anthracyl, and phenanthryl;
one of Y₁ and Y₂ is a carbon, and the other one of Y₁ and Y₂ is oxygen or sulfur;
Ar₁ is selected from a group consisting of a substituted or unsubstituted phenyl, a substituted or unsubstituted naphthyl, a substituted or unsubstituted anthracyl, a substituted or unsubstituted phenanthryl, a substituted or unsubstituted acenaphthylenyl, and a substituted or unsubstituted aromatic heterocyclic group; m is 0 or 1;
D₁ and D₂ are each independently selected from a group consisting of a substituted or unsubstituted phenyl, a substituted or unsubstituted biphenyl, a substituted or unsubstituted naphthyl, a substituted or unsubstituted anthracyl, a substituted or unsubstituted phenanthryl, a substituted or unsubstituted acenaphthylenyl, a substituted or unsubstituted pyrenyl, a substituted or unsubstituted perylenyl, a substituted or unsubstituted fluorenyl, a substituted or unsubstituted spirodifluorenyl, a substituted or unsubstituted chrysenyl, a substituted or unsubstituted benzophenanthryl, a substituted or unsubstituted benzoanthracyl, a substituted or unsubstituted fluoranthenyl, a substituted or unsubstituted picenyl, a substituted or unsubstituted furyl, a substituted or unsubstituted benzofuryl, a substituted or unsubstituted dibenzofuryl, a substituted or unsubstituted thienyl, a substituted or unsubstituted benzothienyl, a substituted or unsubstituted dibenzothienyl, a substituted or unsubstituted phenoxazinyl, a substituted or unsubstituted phenazinyl, a substituted or unsubstituted phenothiazinyl, a substituted or unsubstituted thioxazinyl, a substituted or unsubstituted thianthrenyl, a carbazole-derived group, an acridine-derived group, diarylamino, a diarylamine-derived group, triarylamino and a triarylamine-derived group; and
Ar₂ and Ar₃ are each independently selected from a group consisting of a substituted or unsubstituted phenyl, a substituted or unsubstituted biphenyl, a substituted or unsubstituted naphthyl, a substituted or unsubstituted anthracyl, a substituted or unsubstituted phenanthryl, a substituted or unsubstituted acenaphthylenyl, a substituted or unsubstituted pyrenyl, a substituted or unsubstituted perylenyl, a substituted or unsubstituted fluorenyl, a substituted or unsubstituted spirodifluorenyl, a substituted or unsubstituted chrysenyl, a substituted or unsubstituted benzophenanthryl, a substituted or unsubstituted benzoanthracyl, a substituted or unsubstituted fluoranthenyl, a substituted or unsubstituted picenyl, a substituted or unsubstituted furyl, a substituted or unsubstituted benzofuryl, a substituted or unsubstituted dibenzofuryl, a substituted or unsubstituted thienyl, a substituted or unsubstituted benzothienyl, a substituted or unsubstituted dibenzothienyl, a substituted or unsubstituted phenoxazinyl, a substituted or unsubstituted phenazinyl, a substituted or unsubstituted phenothiazinyl, a substituted or unsubstituted thioxazinyl, a substituted or unsubstituted thianthrenyl, a carbazole-derived group, an acridine-derived group, diarylamino, a diarylamine-derived group, triarylamino and a triarylamine-derived group.

In a second aspect, the present disclosure provides a display panel including an organic light-emitting component. The organic light-emitting component includes an anode, a cathode, a light-emitting layer positioned between the anode and the cathode, and a capping layer covering the cathode. The capping layer includes one or more benzo heterocyclic compounds according to the first aspect of the present disclosure.

In a third aspect, the present disclosure provides a display device including the display panel according to the second aspect of the present disclosure.

### BRIEF DESCRIPTION OF THE DRAWINGS

FIG. 1 is a chemical formula of a benzo heterocyclic compound according to the present disclosure;

FIG. 2 is a structural schematic diagram of an organic light-emitting component according to an embodiment of the present disclosure; and

FIG. 3 is a diagram of a display device of the present disclosure.

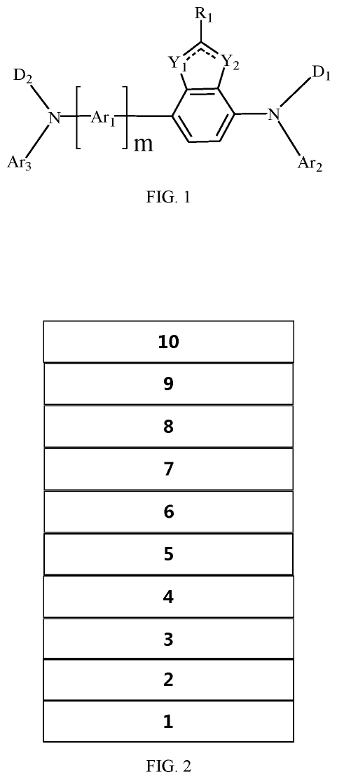

### DESCRIPTION OF EMBODIMENTS

The present disclosure is described in detail with aid of embodiments and comparative examples. These embodiments are only used to illustrate the present disclosure, but not intended to limit the scope of the present disclosure. Without departing from the scope of the present disclosure, any modification or equivalent replacement with respect to the technical solutions of the present disclosure shall fall into the scope of protection of the present disclosure.

In a first aspect, the present disclosure provides a benzo heterocyclic compound having a structure represented by Formula (I):

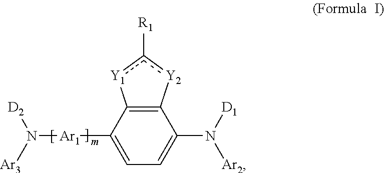

in which R₁ is selected from a group consisting of hydrogen, C1-C20 alkyl, C1-C20 alkoxy, phenyl, naphthyl, anthracyl, and phenanthryl;
one of Y₁ and Y₂ is carbon, and the other one of Y₁ and Y₂ is oxygen or sulfur;
Ar₁ is selected from a group consisting of a substituted or unsubstituted phenyl, a substituted or unsubstituted naphthyl, a substituted or unsubstituted anthracyl, a substituted or unsubstituted phenanthryl, a substituted or unsubstituted acenaphthylenyl, and a substituted or unsubstituted aromatic heterocyclic group; m is 0 or 1;
D₁ and D₂ are each independently selected from a group consisting of a substituted or unsubstituted phenyl, a substituted or unsubstituted biphenyl, a substituted or unsubstituted naphthyl, a substituted or unsubstituted anthracyl, a substituted or unsubstituted phenanthryl, a substituted or unsubstituted acenaphthylenyl, a substituted or unsubstituted pyrenyl, a substituted or unsubstituted perylenyl, a substituted or unsubstituted fluorenyl, a substituted or unsubstituted spirodifluorenyl, a substituted or unsubstituted chrysenyl, a substituted or unsubstituted benzophenanthryl, a substituted or unsubstituted benzoanthracyl, a substituted or unsubstituted fluoranthenyl, a substituted or unsubstituted picenyl, a substituted or unsubstituted furyl, a substituted or unsubstituted benzofuryl, a substituted or unsubstituted dibenzofuryl, a substituted or unsubstituted thienyl, a substituted or unsubstituted benzothienyl, a substituted or unsubstituted dibenzothienyl, a substituted or unsubstituted phenoxazinyl, a substituted or unsubstituted phenazinyl, a substituted or unsubstituted phenothiazinyl, a substituted or unsubstituted thioxazinyl, a substituted or unsubstituted thianthrenyl, a carbazole-derived group, an acridine-derived group, diarylamino, a diarylamine-derived group, triarylamino and a triarylamine-derived group; and
Ar₂ and Ar₃ are each independently selected from a group consisting of a substituted or unsubstituted phenyl, a substituted or unsubstituted biphenyl, a substituted or unsubstituted naphthyl, a substituted or unsubstituted anthracyl, a substituted or unsubstituted phenanthryl, a substituted or unsubstituted acenaphthylenyl, a substituted or unsubstituted pyrenyl, a substituted or unsubstituted perylenyl, a substituted or unsubstituted fluorenyl, a substituted or unsubstituted spirodifluorenyl, a substituted or unsubstituted chrysenyl, a substituted or unsubstituted benzophenanthryl, a substituted or unsubstituted benzoanthracyl, a substituted or unsubstituted fluoranthenyl, a substituted or unsubstituted picenyl, a substituted or unsubstituted furyl, a substituted or unsubstituted benzofuryl, a substituted or unsubstituted dibenzofuryl, a substituted or unsubstituted thienyl, a substituted or unsubstituted benzothienyl, a substituted or unsubstituted dibenzothienyl, a substituted or unsubstituted phenoxazinyl, a substituted or unsubstituted phenazinyl, a substituted or unsubstituted phenothiazinyl, a substituted or unsubstituted thioxazinyl, a substituted or unsubstituted thianthrenyl, a carbazole-derived group, an acridine-derived group, diarylamino, a diarylamine-derived group, triarylamino and a triarylamine-derived group.

At least the following advantages can be obtained by using the benzo heterocyclic compound according to the present disclosure:
(1) the benzo heterocyclic compound according to the present disclosure contains oxygen or sulfur element (especially sulfur element), and thus has a high polarizability, which is conducive to obtaining materials with high refractive index;
(2) the benzo heterocyclic compound according to the present disclosure has unique electrical, optical, redox and self-assembly properties, and thus a more ordered molecular alignment can be achieved, making it an important candidate for organic electronics materials;
(3) oxygen atom or sulfur atom has good polarizability and electron-rich properties, and in this regard, this kind of aromatic heterocyclic organic materials has good charge transfer and electron-donating properties.

By using the benzothiophene or benzopyrrole compounds of the present disclosure as the CPL material of the organic light-emitting component, the high light extraction efficiency can be obtained, and the problem of color purity reduction of the blue light-emitting devices can be avoided.

In an embodiment of the benzo heterocyclic compound according to the present disclosure, the benzo heterocyclic compound has a structure represented by Formula (I-1):

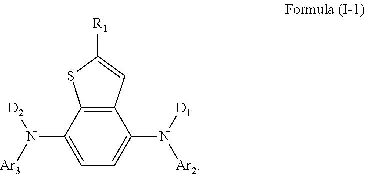

In an embodiment of the benzo heterocyclic compound according to the present disclosure, the benzo heterocyclic compound has a structure represented by Formula (I-2):

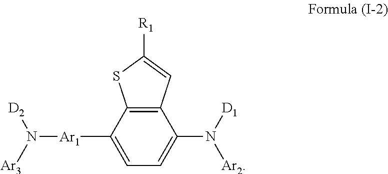

In an embodiment of the benzo heterocyclic compound according to the present disclosure, the benzo heterocyclic compound has a structure represented by Formula (I-3):

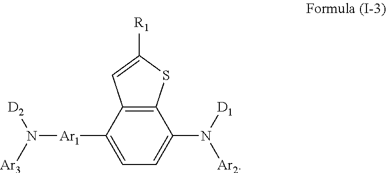

Benzothiophene moiety has a large rigid planar symmetric conjugate structure, which enhances delocalization ability of π electrons and promotes π-π interaction of molecules, facilitating a compact molecular stacking and charge transfer. Benzothiophene moiety is easy to be chemically modified, easy to be synthesized and purified, and has high stability in air environment.

Benzothiophene moiety can be modified by incorporating different types of substituents through chemical tailoring, so as to adjust solubility and energy levels of the whole molecule, and its planar symmetric structure can effectively enhance the π-π stacking between molecular chains, so as to obtain higher hole mobility.

In the benzo heterocyclic compound according to the present disclosure, the substituents are all located on the aromatic rings, without prolong a significant length of the conjugated main chain, and a red shift of absorption spectrum is non-significant. In addition, the introduction of thiophene ring can improve the refractive index without a remarkable increase in absorption of blue light in the absorption spectra. Therefore, the benzo heterocyclic compound according to the present disclosure is suitable to be as the CPL material for OLED components.

In an embodiment of the benzo heterocyclic compound according to the present disclosure, the aromatic heterocyclic group is selected from a group consisting of thienyl, thiazolyl, thiadiazolyl, furyl, oxazolyl, and oxadiazolyl.

In an embodiment of the benzo heterocyclic compound according to the present disclosure, D₁ and D₂ are each independently selected from a group consisting of a carbazole-derived group, an acridine-derived group, diarylamino, a diarylamine-derived group, triarylamino and a triarylamine-derived group; and Ar₂ and Ar₃ are each independently selected from a group consisting of a substituted or unsubstituted phenyl, a substituted or unsubstituted biphenyl, a substituted or unsubstituted naphthyl, and a substituted or unsubstituted anthracyl.

In an embodiment of the benzo heterocyclic compound according to the present disclosure, the carbazole-derived group is selected from a group consisting of the following groups:

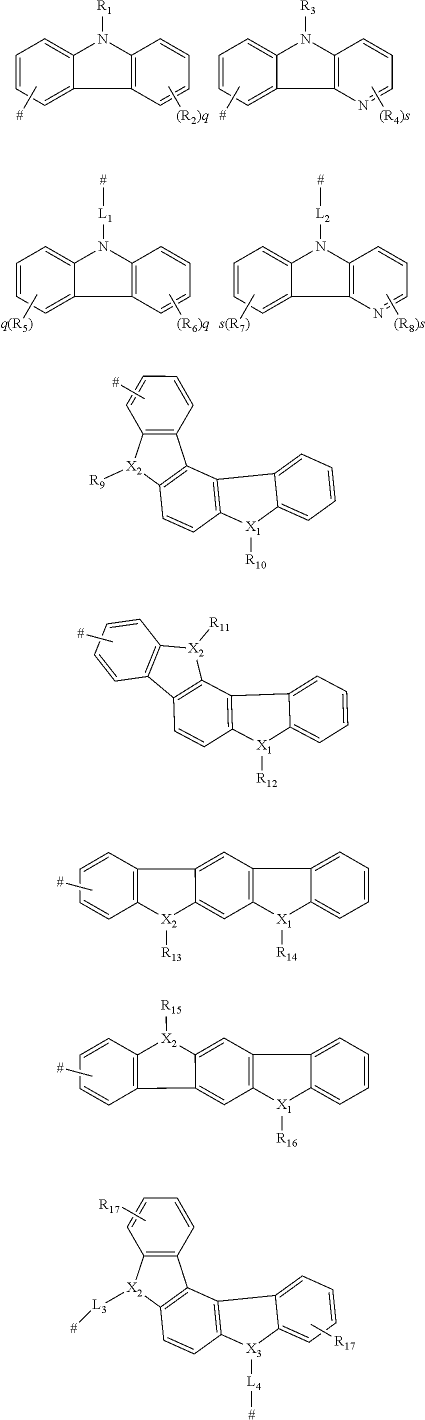

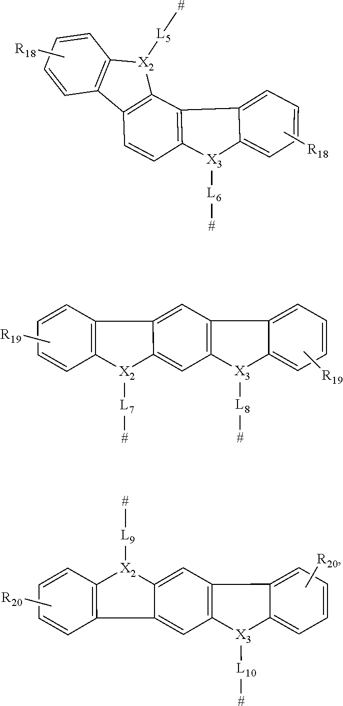

in which X₁, X₂, and X₃ are each independently selected from a group consisting of carbon, nitrogen, oxygen, sulfur, and silicon, and at least one of X₁, X₂, and X₃ is nitrogen;
R₁-R₂₀ are each independently selected from a group consisting of alkyl, alkoxy, an aromatic ring group, an aromatic fused ring group, and an aromatic heterocyclic group; q and s are integers independently selected from 0, 1, 2, and 3;
L₁-L₁₀ are each independently selected from a group consisting of an aromatic ring group, an aromatic fused ring group, and an aromatic heterocyclic group; and
# represents a bonding position.

In an embodiment of the benzo heterocyclic compound according to the present disclosure, the aromatic ring group is phenyl; the aromatic fused ring group is selected from a group consisting of naphthyl, anthracyl, pyrenyl, perylenyl; and the aromatic heterocyclic group is selected from a group consisting of thienyl, furyl, and thiazolyl.

In an embodiment of the benzo heterocyclic compound according to the present disclosure, the carbazole-derived group is selected from a group consisting of the following groups:

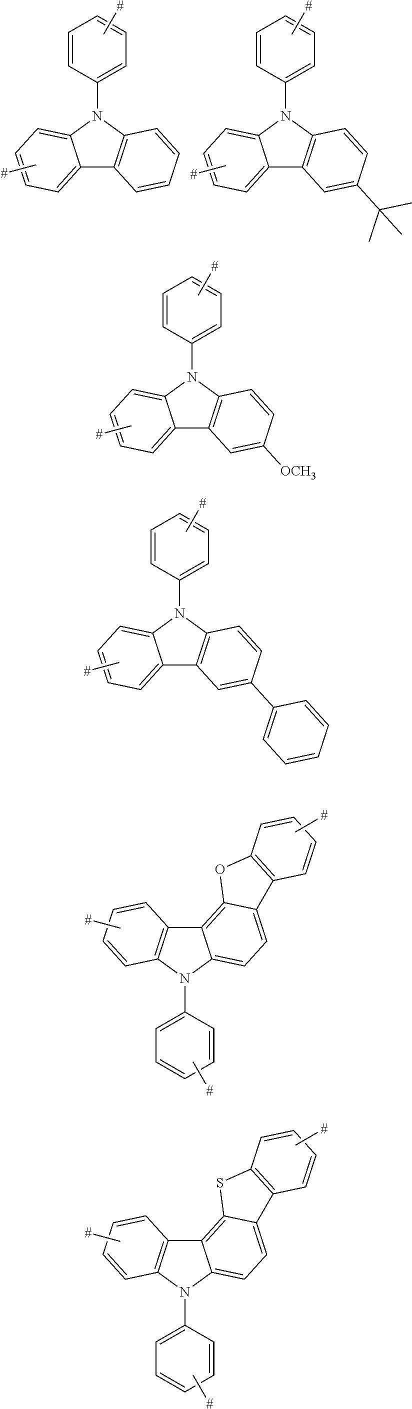

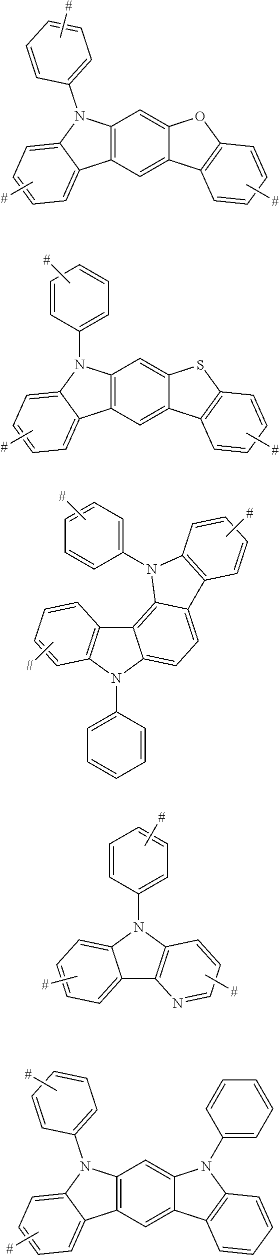

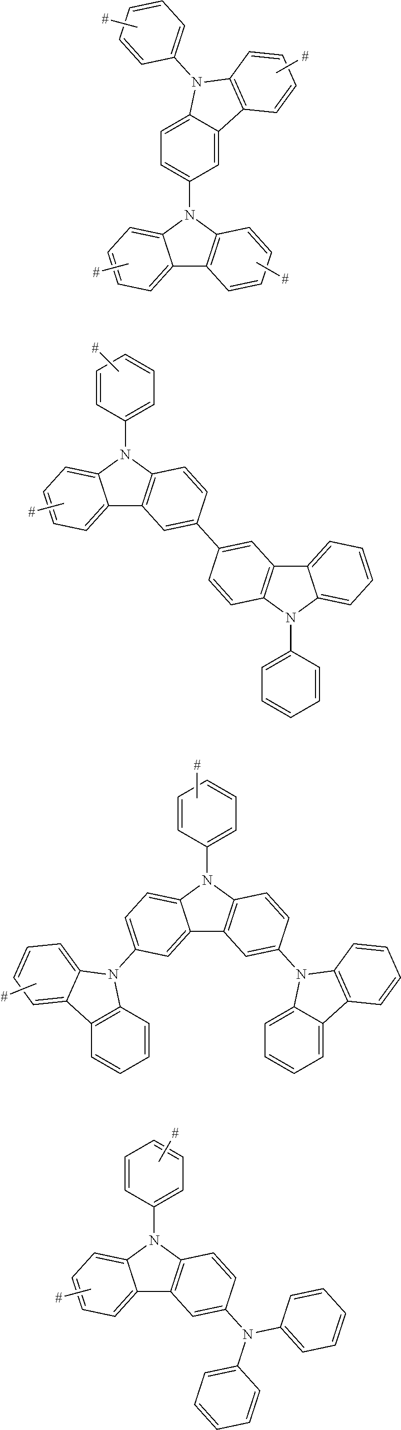

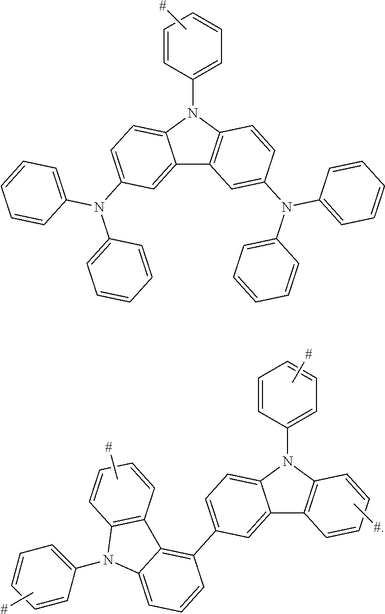

The carbazolyl, as an electron-donating group, can form a dipole-dipole interaction together with the central benzo heterocyclic ring, which enhances polarizability and makes the compound according to the present disclosure have a higher refractive index. Carbazole can be evaporated in high vacuum to form an amorphous film without pinhole defects, which has better thermal, chemical, electrochemical and photochemical stability. Raw material is accessible, cheap, and easy to be synthesized.

In an embodiment of the benzo heterocyclic compound according to the present disclosure, the acridine-derived group is selected from a group consisting of the following groups:

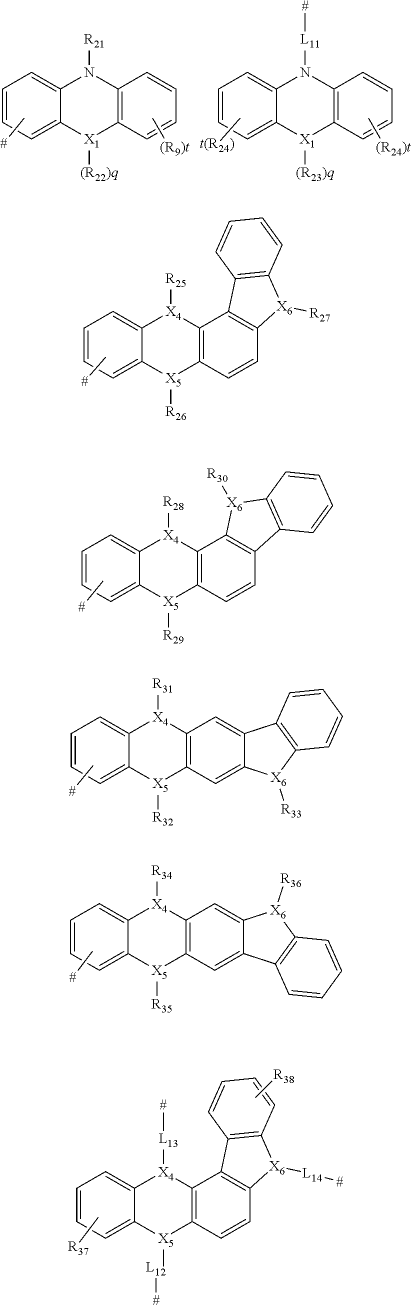

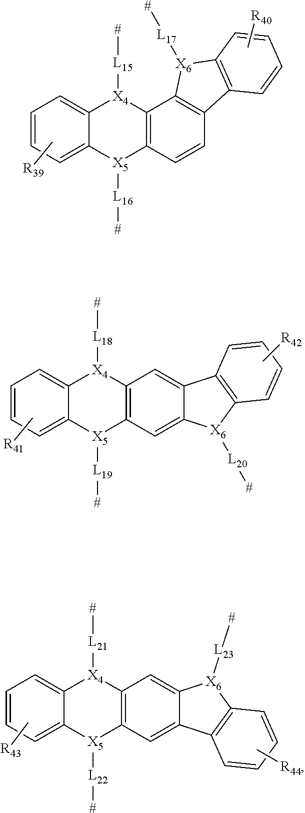

in which X₁, X₄, X₅, and X₆ are each independently selected from a group consisting of carbon, nitrogen, oxygen, sulfur, and silicon, and at least one of X₄ and X₅ is nitrogen;
R₂₁-R₄₄ are each independently selected from a group consisting of alkyl, alkoxy, an aromatic ring group, an aromatic fused ring group, and an aromatic heterocyclic group, q and t are integers independently selected from 0, 1, 2, and 3;
L₁₁-L₂₃ are each independently selected from a group consisting of an aromatic ring group, an aromatic fused ring group, and an aromatic heterocyclic group; and
# represents a bonding position.

In an embodiment of the benzo heterocyclic compound according to the present disclosure, the acridine-derived group is selected from a group consisting of the following groups:

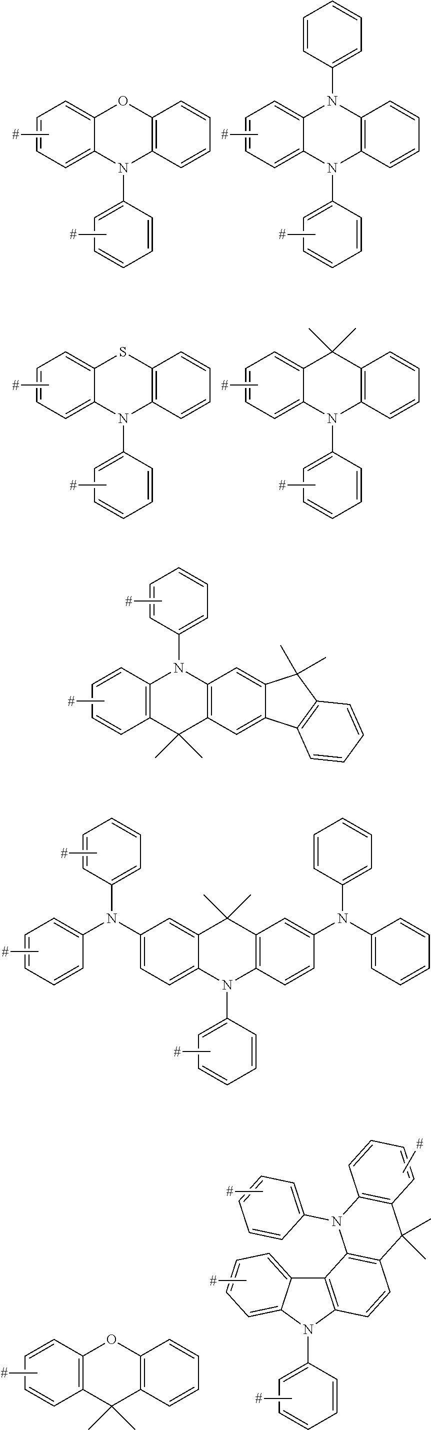

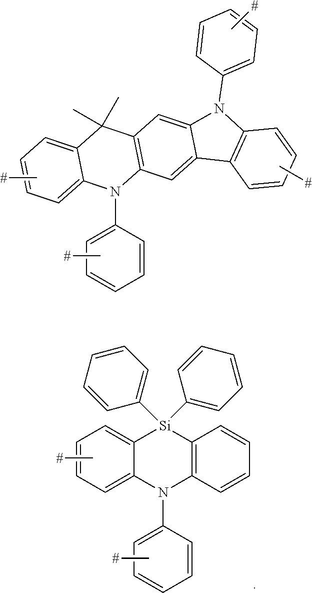

The acridine-derived groups are stronger electron-donating groups than the carbazole-derived groups. The acridine-derived groups can form stronger dipole-dipole interaction together with the central benzo heterocyclic ring, which further enhances the polarizability and makes the compound according to the present disclosure have a higher refractive index. The N atom on the acridine-derived group is in an sp3 hybrid state, and has a non-planar structure, which is conducive to forming a uniform amorphous film by vapor deposition. At the same time, the acridine-derived group has a plurality of linkable sites, facilitating a chemical modification.

In an embodiment of the benzo heterocyclic compound according to the present disclosure, the diarylamine group or the triarylamine group is selected from a group consisting of the following groups:

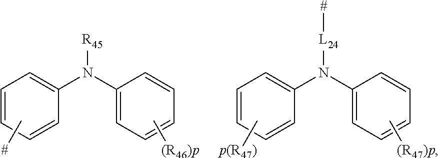

in which R₄₅-R₄₇ are each independently selected from a group consisting of alkyl, alkoxy, an aromatic ring group, an aromatic fused ring group, and an aromatic heterocyclic group; p is an integer selected from 0, 1, 2, and 3;
L₂₄ is selected from a group consisting of an aromatic ring group, an aromatic fused ring group, and an aromatic heterocyclic group; and
# represents a bonding position.

In an embodiment of the benzo heterocyclic compound according to the present disclosure, the diarylamine group or the triarylamine group is selected from a group consisting of the following groups:

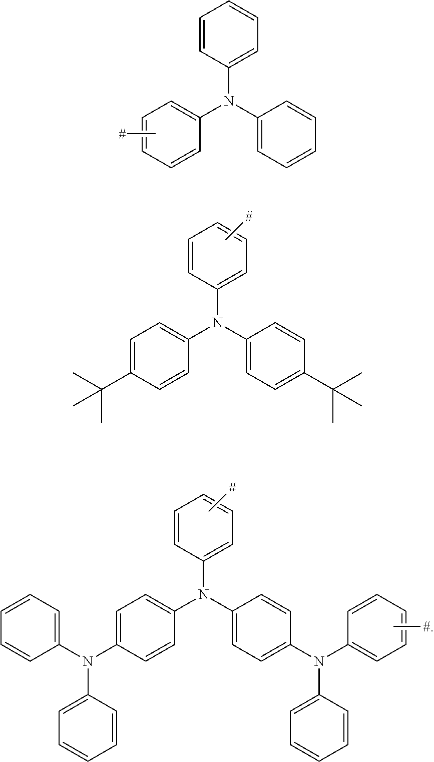

Aromatic amine-derived groups have the following advantages: (1) the aromatic amine-derived groups are typical electron-donating groups with moderate electron-donating property, and can form strong dipole-dipole interaction together with the central benzo heterocyclic ring, which enhances the polarizability and makes the compound according to the present disclosure have a high refractive index; (2) the aromatic amine-derived groups have good thermal, electrochemical and photochemical stability; the aromatic amine-derived material is accessible, cheap, and easy to be chemically modified; (3) N atom on the aromatic amine-derived group is in sp3 hybrid state, and has a non-planar structure, which is conducive to forming a uniform amorphous film.

In an embodiment of the benzo heterocyclic compound according to the present disclosure, D₁ and D₂ are each independently selected from a group consisting of the following groups:

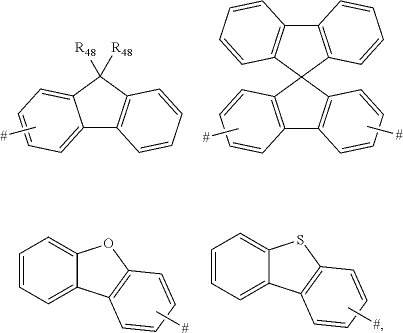

in which R₄₈ is selected from a group consisting of alkyl, alkoxy, an aromatic ring group, an aromatic fused ring group, and an aromatic heterocyclic group.

Fluorenyl has the following application advantages: (1) fluorenyl has a rigid planar structure, sites 2, 7 and 9 on the fluorenyl are accessible to chemical modification, such as incorporating various functional groups, for modifying physical and chemical properties, such as optical, electrical and processing properties of the molecules; (2) fluorenyl has a weak absorption in the visible light region, and is suitable to be used as the material of the capping layer; (3) fluorenyl has high thermal, electrochemical and photochemical stability; (4) the material of fluorenyl is cheap and easy to get.

Spirodifluorenyl bridges two fluorene monomers with a carbon atom in an sp3 hybrid state as the center, such that the two fluorene monomers have a non-planar spatial orthogonal structure, which can avoid a compact stacking of molecules and the formation of aggregates in the film. It is conducive to forming more stable and uniform amorphous film structure and improving the thermal stability of the material, thereby effectively increasing the glass transition temperature of the material, and inhibiting molecular crystallization. In this way, a more stable amorphous material can be obtained. The spatial orthogonal structure is also conducive to improving the solubility of the compound and facilitating solution processing. The structure of spirodifluorenyl has many reactive sites that can be chemically modified, and thus is also conducive to expanding a three-dimensional topological structure.

In an embodiment of the benzo heterocyclic compound according to the present disclosure, the benzo heterocyclic compound is selected from a group consisting of the following compounds:

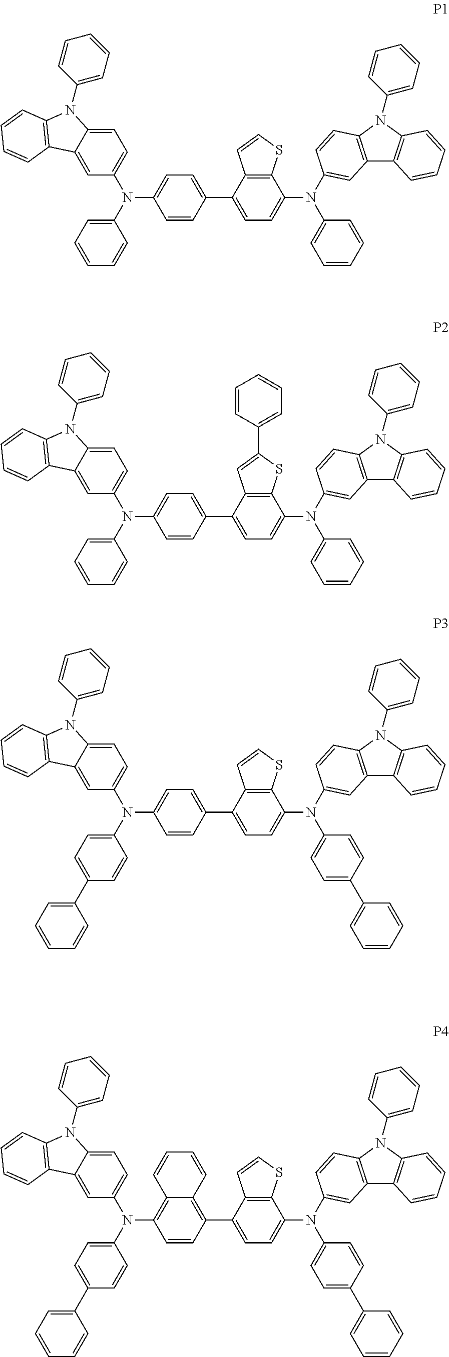

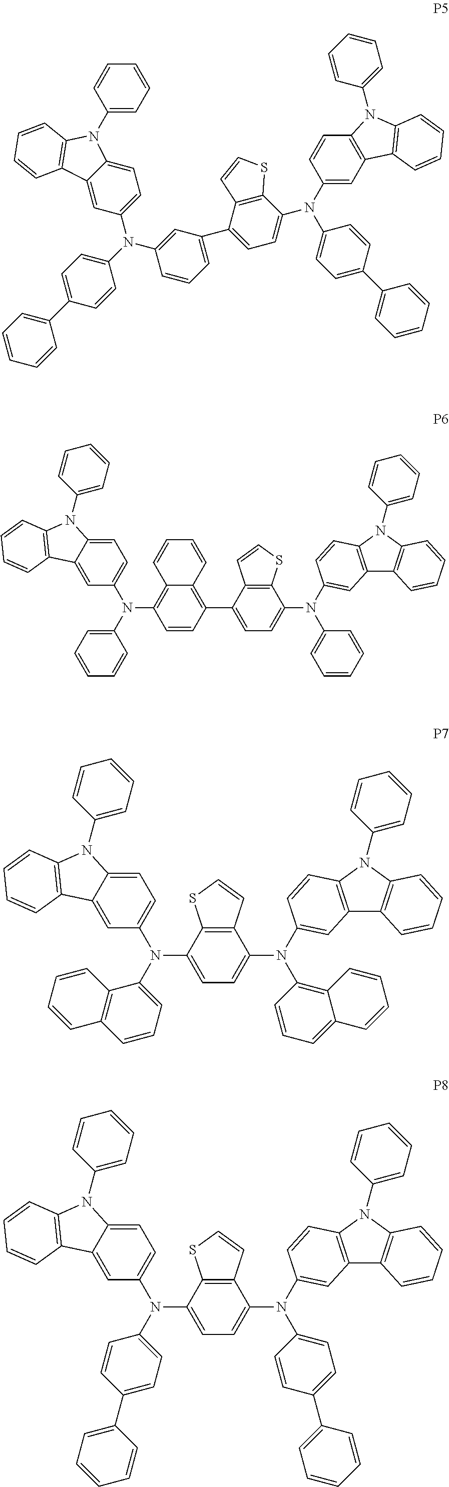 [SMILES: c1ccc(-c2ccc(N(c3cccc(-c4ccc(N(c5ccc(-c6ccccc6)cc5)c5ccc6c(c5)c5ccccc5n6-c5ccccc5)c5sccc45)c3)c3ccc4c(c3)c3ccccc3n4-c3ccccc3)cc2)cc1 ; c1ccc(N(c2ccc3c(c2)c2ccccc2n3-c2ccccc2)c2ccc(-c3ccc(N(c4ccccc4)c4ccc5c(c4)c4ccccc4n5-c4ccccc4)c4sccc34)c3ccccc23)cc1 ; c1ccc(-n2c3ccccc3c3cc(N(c4cccc5ccccc45)c4ccc(N(c5ccc6c(c5)c5ccccc5n6-c5ccccc5)c5cccc6ccccc56)c5sccc45)ccc32)cc1 ; c1ccc(-c2ccc(N(c3ccc4c(c3)c3ccccc3n4-c3ccccc3)c3ccc(N(c4ccc(-c5ccccc5)cc4)c4ccc5c(c4)c4ccccc4n5-c4ccccc4)c4sccc34)cc2)cc1]

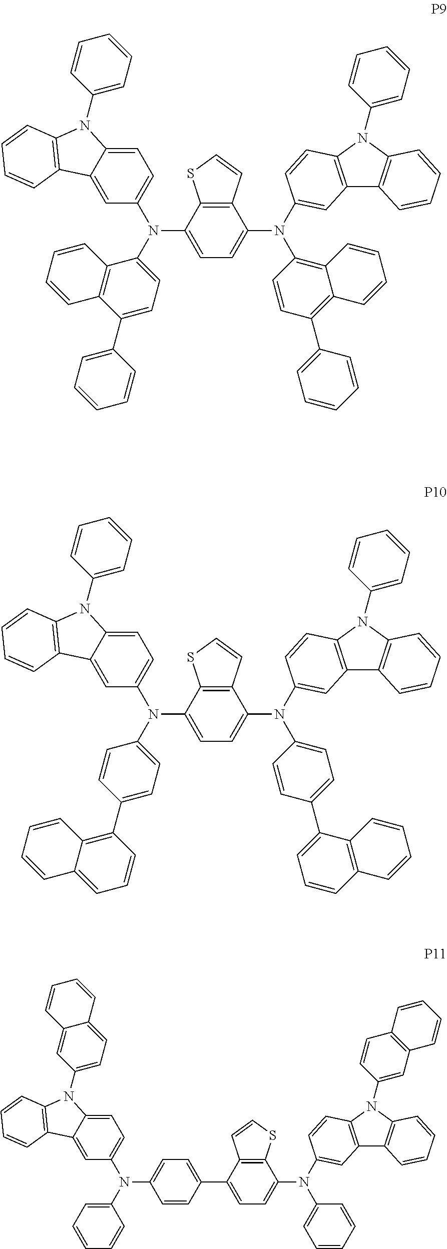 [SMILES: c1ccc(-c2ccc(N(c3ccc4c(c3)c3ccccc3n4-c3ccccc3)c3ccc(N(c4ccc5c(c4)c4ccccc4n5-c4ccccc4)c4ccc(-c5ccccc5)c5ccccc45)c4sccc34)c3ccccc23)cc1 ; c1ccc(-n2c3ccccc3c3cc(N(c4ccc(-c5cccc6ccccc56)cc4)c4ccc(N(c5ccc(-c6cccc7ccccc67)cc5)c5ccc6c(c5)c5ccccc5n6-c5ccccc5)c5sccc45)ccc32)cc1 ; c1ccc(N(c2ccc(-c3ccc(N(c4ccccc4)c4ccc5c(c4)c4ccccc4n5-c4ccc5ccccc5c4)c4sccc34)cc2)c2ccc3c(c2)c2ccccc2n3-c2ccc3ccccc3c2)cc1]

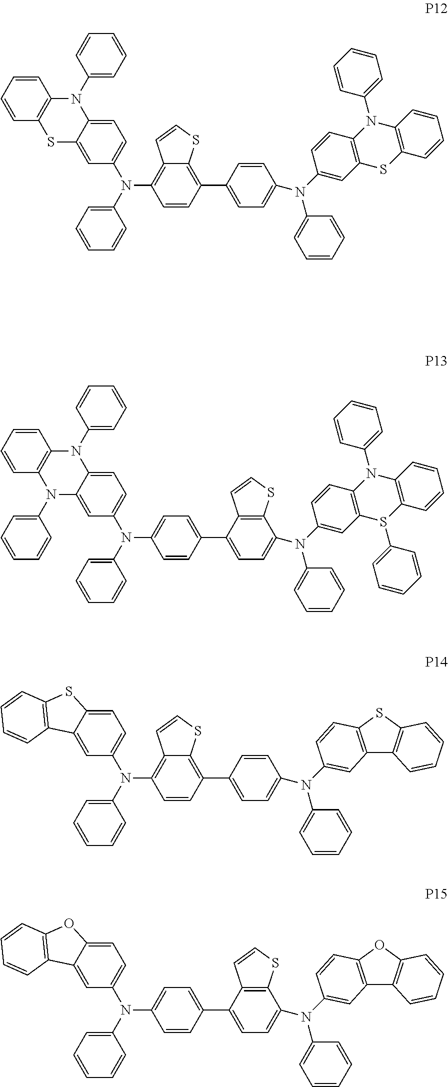

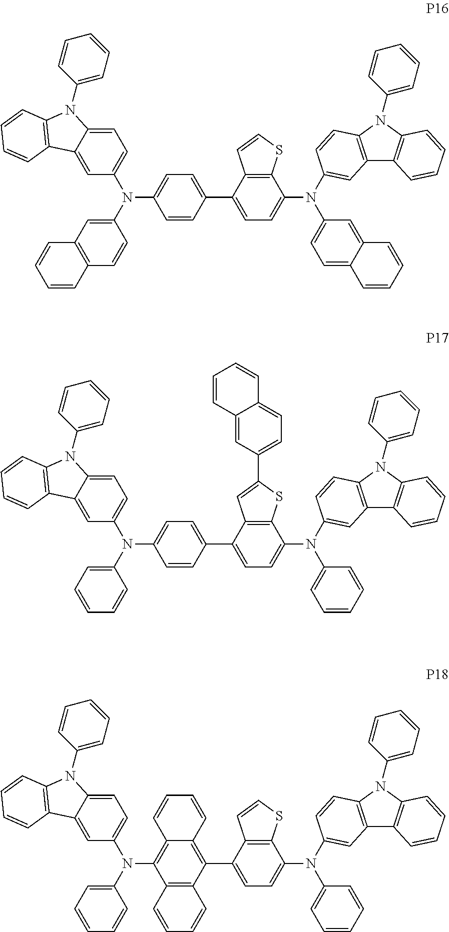 [SMILES: c1ccc(-n2c3ccccc3c3cc(N(c4ccc(-c5ccc(N(c6ccc7ccccc7c6)c6ccc7c(c6)c6ccccc6n7-c6ccccc6)c6sccc56)cc4)c4ccc5ccccc5c4)ccc32)cc1 ; c1ccc(N(c2ccc(-c3ccc(N(c4ccccc4)c4ccc5c(c4)c4ccccc4n5-c4ccccc4)c4sc(-c5ccc6ccccc6c5)cc34)cc2)c2ccc3c(c2)c2ccccc2n3-c2ccccc2)cc1 ; c1ccc(N(c2ccc3c(c2)c2ccccc2n3-c2ccccc2)c2c3ccccc3c(-c3ccc(N(c4ccccc4)c4ccc5c(c4)c4ccccc4n5-c4ccccc4)c4sccc34)c3ccccc23)cc1]

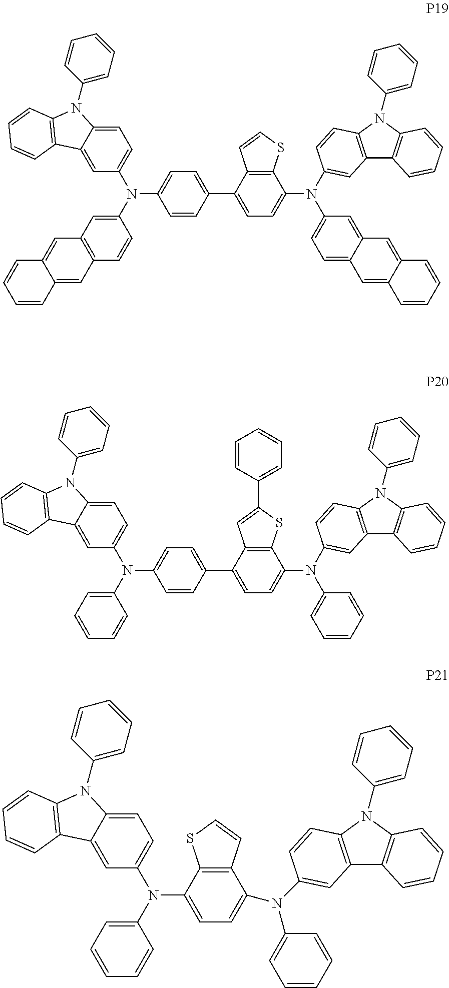 [SMILES: c1ccc(-n2c3ccccc3c3cc(N(c4ccc(-c5ccc(N(c6ccc7cc8ccccc8cc7c6)c6ccc7c(c6)c6ccccc6n7-c6ccccc6)c6sccc56)cc4)c4ccc5cc6ccccc6cc5c4)ccc32)cc1 ; c1ccc(-c2cc3c(-c4ccc(N(c5ccccc5)c5ccc6c(c5)c5ccccc5n6-c5ccccc5)cc4)ccc(N(c4ccccc4)c4ccc5c(c4)c4ccccc4n5-c4ccccc4)c3s2)cc1 ; c1ccc(N(c2ccc3c(c2)c2ccccc2n3-c2ccccc2)c2ccc(N(c3ccccc3)c3ccc4c(c3)c3ccccc3n4-c3ccccc3)c3sccc23)cc1]

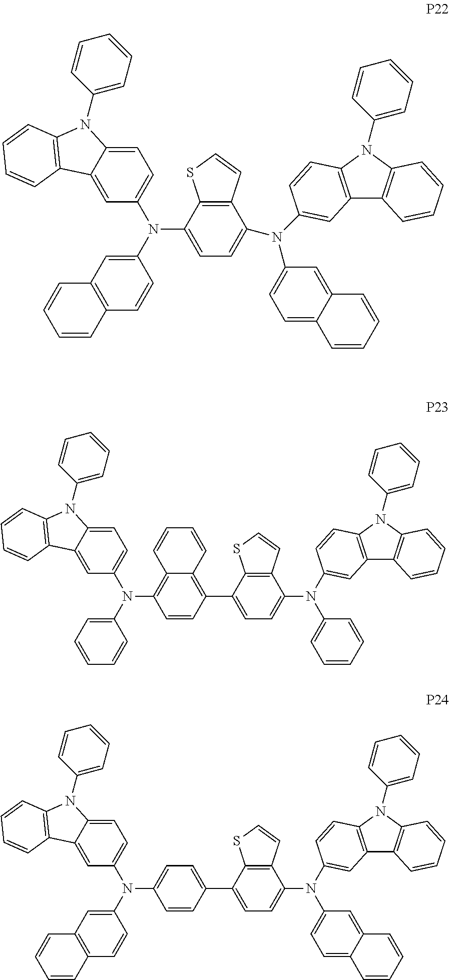 [SMILES: c1ccc(-n2c3ccccc3c3cc(N(c4ccc5ccccc5c4)c4ccc(N(c5ccc6ccccc6c5)c5ccc6c(c5)c5ccccc5n6-c5ccccc5)c5sccc45)ccc32)cc1 ; c1ccc(N(c2ccc3c(c2)c2ccccc2n3-c2ccccc2)c2ccc(-c3ccc(N(c4ccccc4)c4ccc5c(c4)c4ccccc4n5-c4ccccc4)c4ccsc34)c3ccccc23)cc1 ; c1ccc(-n2c3ccccc3c3cc(N(c4ccc(-c5ccc(N(c6ccc7ccccc7c6)c6ccc7c(c6)c6ccccc6n7-c6ccccc6)c6ccsc56)cc4)c4ccc5ccccc5c4)ccc32)cc1]

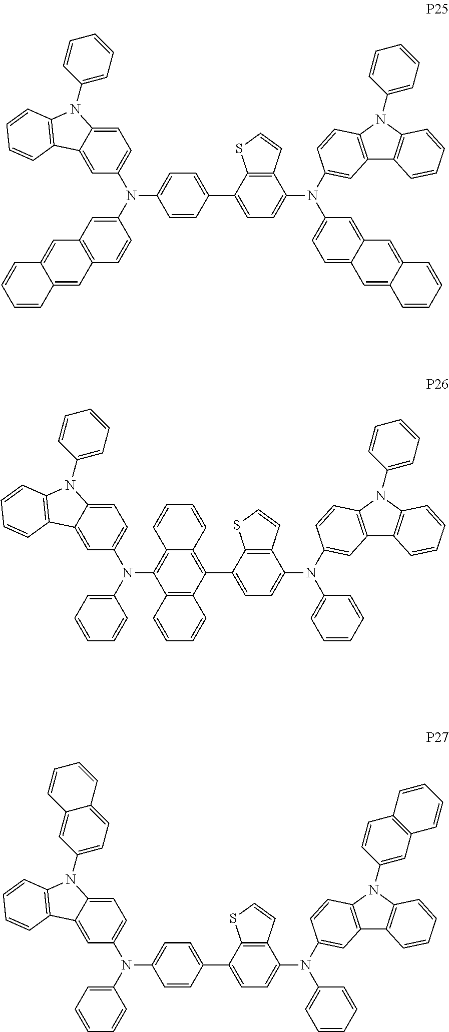 [SMILES: c1ccc(-n2c3ccccc3c3cc(N(c4ccc(-c5ccc(N(c6ccc7cc8ccccc8cc7c6)c6ccc7c(c6)c6ccccc6n7-c6ccccc6)c6ccsc56)cc4)c4ccc5cc6ccccc6cc5c4)ccc32)cc1 ; c1ccc(N(c2ccc3c(c2)c2ccccc2n3-c2ccccc2)c2c3ccccc3c(-c3ccc(N(c4ccccc4)c4ccc5c(c4)c4ccccc4n5-c4ccccc4)c4ccsc34)c3ccccc23)cc1 ; c1ccc(N(c2ccc(-c3ccc(N(c4ccccc4)c4ccc5c(c4)c4ccccc4n5-c4ccc5ccccc5c4)c4ccsc34)cc2)c2ccc3c(c2)c2ccccc2n3-c2ccc3ccccc3c2)cc1]

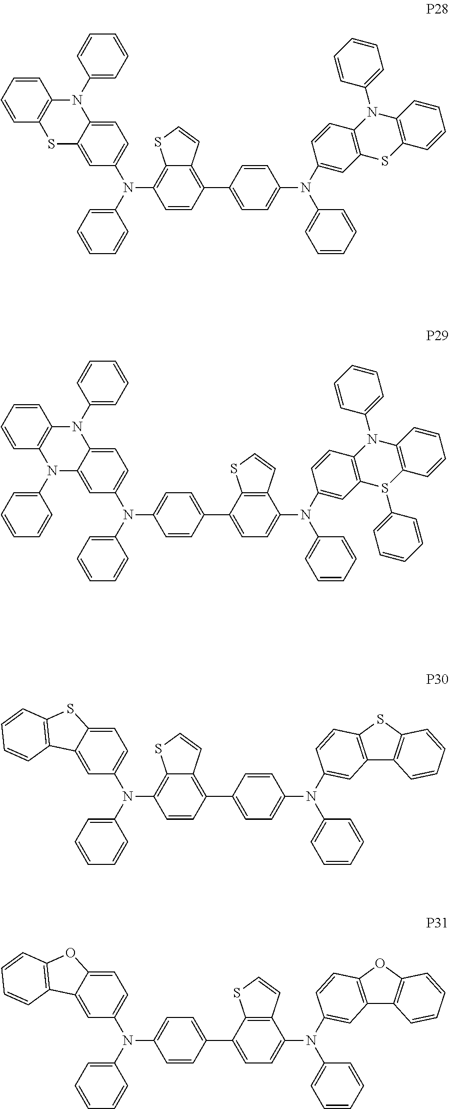

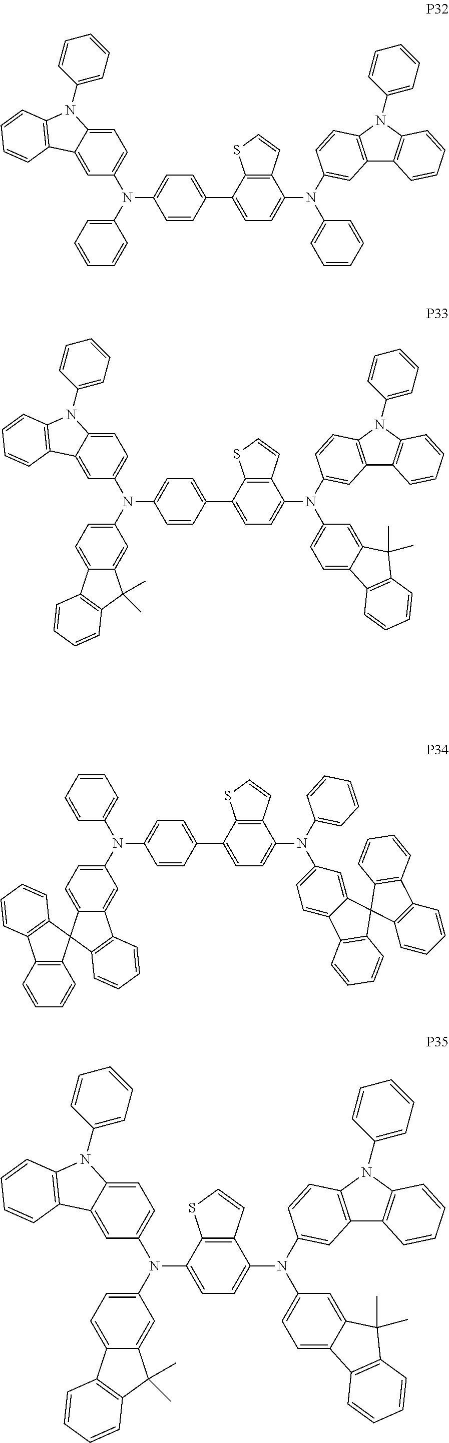

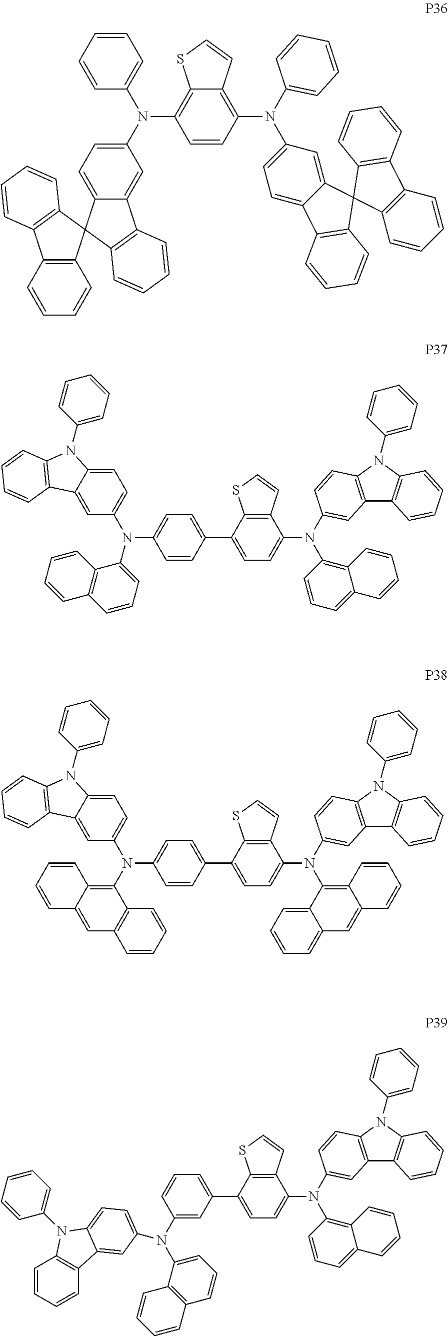

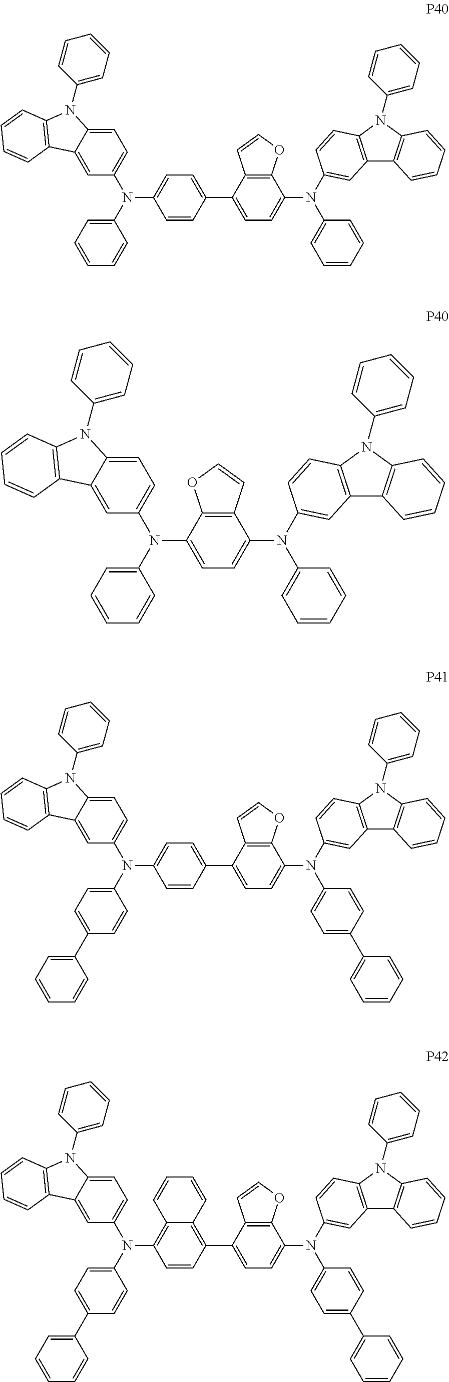 [SMILES: c1ccc(N(c2ccc(-c3ccc(N(c4ccccc4)c4ccc5c(c4)c4ccccc4n5-c4ccccc4)c4occc34)cc2)c2ccc3c(c2)c2ccccc2n3-c2ccccc2)cc1 ; c1ccc(N(c2ccc3c(c2)c2ccccc2n3-c2ccccc2)c2ccc(N(c3ccccc3)c3ccc4c(c3)c3ccccc3n4-c3ccccc3)c3occc23)cc1 ; c1ccc(-c2ccc(N(c3ccc(-c4ccc(N(c5ccc(-c6ccccc6)cc5)c5ccc6c(c5)c5ccccc5n6-c5ccccc5)c5occc45)cc3)c3ccc4c(c3)c3ccccc3n4-c3ccccc3)cc2)cc1 ; c1ccc(-c2ccc(N(c3ccc4c(c3)c3ccccc3n4-c3ccccc3)c3ccc(-c4ccc(N(c5ccc(-c6ccccc6)cc5)c5ccc6c(c5)c5ccccc5n6-c5ccccc5)c5occc45)c4ccccc34)cc2)cc1]

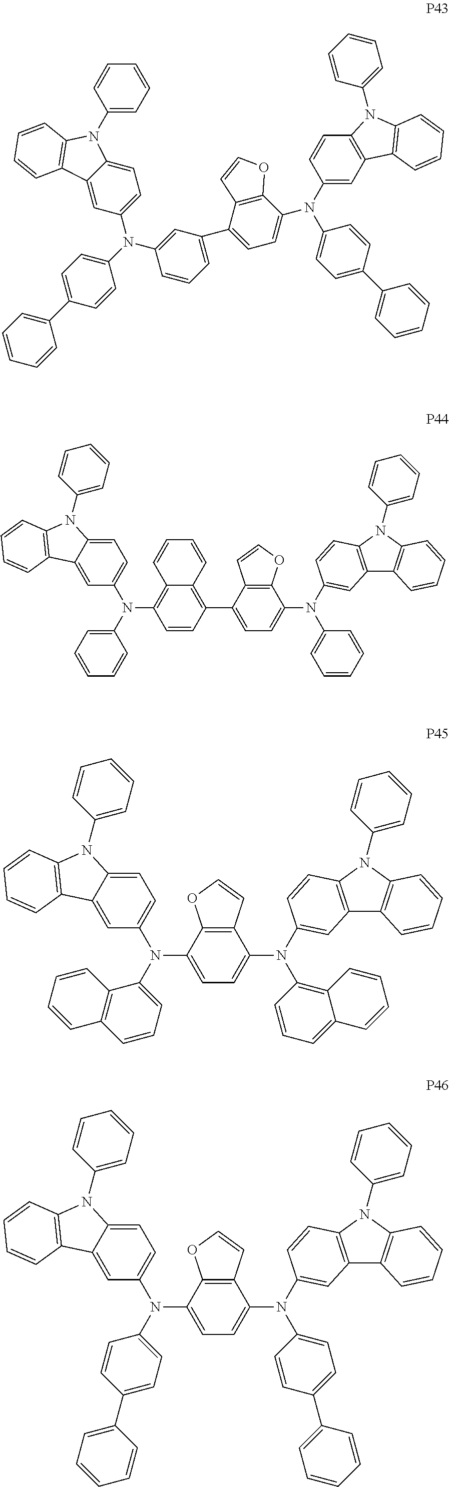 [SMILES: c1ccc(-c2ccc(N(c3cccc(-c4ccc(N(c5ccc(-c6ccccc6)cc5)c5ccc6c(c5)c5ccccc5n6-c5ccccc5)c5occc45)c3)c3ccc4c(c3)c3ccccc3n4-c3ccccc3)cc2)cc1 ; c1ccc(N(c2ccc3c(c2)c2ccccc2n3-c2ccccc2)c2ccc(-c3ccc(N(c4ccccc4)c4ccc5c(c4)c4ccccc4n5-c4ccccc4)c4occc34)c3ccccc23)cc1 ; c1ccc(-n2c3ccccc3c3cc(N(c4cccc5ccccc45)c4ccc(N(c5ccc6c(c5)c5ccccc5n6-c5ccccc5)c5cccc6ccccc56)c5occc45)ccc32)cc1 ; c1ccc(-c2ccc(N(c3ccc4c(c3)c3ccccc3n4-c3ccccc3)c3ccc(N(c4ccc(-c5ccccc5)cc4)c4ccc5c(c4)c4ccccc4n5-c4ccccc4)c4occc34)cc2)cc1]

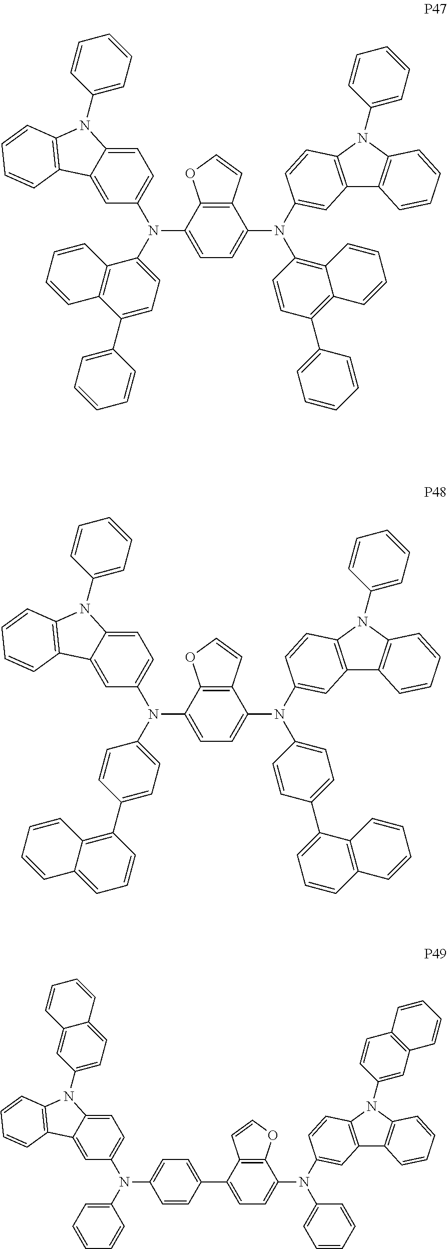 [SMILES: c1ccc(-c2ccc(N(c3ccc4c(c3)c3ccccc3n4-c3ccccc3)c3ccc(N(c4ccc5c(c4)c4ccccc4n5-c4ccccc4)c4ccc(-c5ccccc5)c5ccccc45)c4occc34)c3ccccc23)cc1 ; c1ccc(-n2c3ccccc3c3cc(N(c4ccc(-c5cccc6ccccc56)cc4)c4ccc(N(c5ccc(-c6cccc7ccccc67)cc5)c5ccc6c(c5)c5ccccc5n6-c5ccccc5)c5occc45)ccc32)cc1 ; c1ccc(N(c2ccc(-c3ccc(N(c4ccccc4)c4ccc5c(c4)c4ccccc4n5-c4ccc5ccccc5c4)c4occc34)cc2)c2ccc3c(c2)c2ccccc2n3-c2ccc3ccccc3c2)cc1]

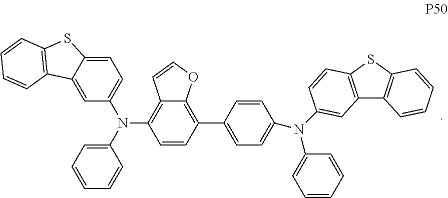 [SMILES: c1ccc(N(c2ccc(-c3ccc(N(c4ccccc4)c4ccc5sc6ccccc6c5c4)c4ccoc34)cc2)c2ccc3sc4ccccc4c3c2)cc1]

For visible light having wavelength of 450 nm-630 nm, the benzo heterocyclic compound according to the present disclosure has a refractive index of 1.85-2.50 and a light extinction coefficient less than 0.1. A difference between refractive indexes of the benzo heterocyclic compound for a visible light of 450 nm and a visible light of 550 nm is smaller than 0.35; and a difference between refractive indexes of the benzo heterocyclic compound for the visible light of 550 nm and a visible light of 630 nm is smaller than 0.15.

The material of the capping layer (CPL) according to the present disclosure has a refractive index decreasing with wavelength in a range of 450-630 nm, and also has a small difference between the refractive indexes for light in a long-wavelength range and light in a short-wavelength range. That is, the refractive indexes for the three colors R/G/B alter in a small range, thereby reducing adverse influence on light extraction efficiency of light having different colors R/G/B. Thus, the material has strong practicability on the optical display panel.

A second aspect of the present disclosure provides an organic light-emitting display panel 11. The organic light-emitting display panel 11 includes an organic light-emitting component. The organic light-emitting component includes an anode 2, a cathode 9, a light-emitting layer 6 positioned between the anode 2 and the cathode 9, and a capping layer (CPL) 10 covering the cathode. The capping layer 10 includes one or more benzo heterocyclic compounds according to the present disclosure.

In an embodiment of the organic light-emitting display panel according to the present disclosure, the capping layer (CPL) has a thickness of 30 nm-100 nm.

In an embodiment of the organic light-emitting display panel according to the present disclosure, a transmittance of the cathode together with the capping layer (CPL) for a visible light of 450-630 nm is greater than 65%.

In an embodiment of the organic light-emitting display panel according to the present disclosure, the organic light-emitting component further includes one or more of a hole injection layer, a hole transmission layer, an electron blocking layer, a hole blocking layer, an electron hole transmission layer, and an electron hole injection layer.

In an embodiment of the organic light-emitting display panel according to the present disclosure, the organic light-emitting component further includes a hole transmission layer, and the hole transmission layer includes one or more benzo heterocyclic compounds according to the present disclosure.

In an embodiment of the organic light-emitting display panel according to the present disclosure, the organic light-emitting component further includes an auxiliary hole transmission layer positioned between the light-emitting layer and the hole transmission layer, and the auxiliary hole transmission layer is configured to control a resonant period of light emitted from the light-emitting layer.

In an embodiment of the organic light-emitting display panel according to the present disclosure, the light-emitting layer includes one or more benzo heterocyclic compounds of the present disclosure.

In an organic light-emitting component provided by the present disclosure, the anode is made of metals such as copper, gold, silver, iron, chromium, nickel, manganese, palladium, platinum, or alloys thereof. The anode can also be made of metal oxides, for example, indium oxide, zinc oxide, indium tin oxide (ITO), indium zinc oxide (IZO), etc. The anode can also be made of conductive polymer such as polyaniline, polypyrrole, poly (3-methylthiophene), or the like. In addition to the anode material mentioned above, the anode also can be made of any suitable material selected from the anode materials known in the related art, and combinations thereof, as long as the material of the anode is conductive to injecting holes.

In the organic light-emitting component according to the present disclosure, the cathode can be made of metals such as aluminum, magnesium, silver, indium, tin, titanium, and alloys thereof. The cathode can also be made of multiple-layer metal material, such as LiF/Al, LiO₂/Al, BaF₂/Al, and the like. In addition to the cathode materials listed above, the cathode also can be made of any suitable material selected from the cathode material known in the related art, and combinations thereof, as long as the material of the cathode is conductive to injecting holes.

In the present disclosure, the organic light-emitting component can be prepared as follows: an anode is formed on a smooth transparent or opaque substrate, a thin organic layer is formed on the anode, and a cathode is formed on the thin organic layer. The thin organic layer can be formed by a known film forming method, for example, vapor deposition, sputtering, spin coating, dipping, ion plating, etc. Finally, an organic capping layer (CPL) is formed on the cathode. The CPL is made of a benzo heterocyclic compound according to the present disclosure. The capping layer (CPL) can be prepared by vapor deposition or a solution processing method. The solution processing method includes ink-jet printing, spin coating, blade coating, screen printing, roll-to-roll printing, and the like.

In another aspect, the present disclosure provides several illustrative methods for preparing the benzo heterocyclic compound according to the present disclosure, as described in the following Examples 1-5.

### Example 1

Synthesis of Compound P1:

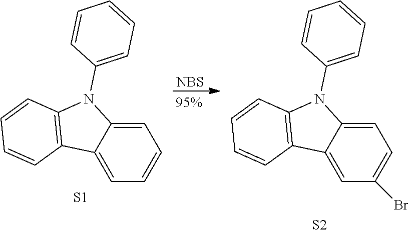

Compound S1 (10 mmol) was added to a two-necked flask (100 mL) and then 30 mL toluene degassed with nitrogen was added to dissolve the Compound S1. One of the two necks of the flask was connected to a constant pressure dropping funnel, and gas in the reaction system was replaced by nitrogen. N-bromosuccimide (NBS) was weighed (5 mmol) and dissolved by adding 20 mL toluene, then the toluene solution of NBS was added dropwise to the toluene solution of the Compound S1 through the dropping funnel at 0° C. and in dark place. After stirring for 2 hours, the temperature of the reaction system was slowly raised to room temperature and stirred overnight. 50 mL deionized water was added to quench the reaction, the mixture was extracted by dichloromethane (100 mL×3), following by collecting the organic phases of extraction and drying with anhydrous Na₂SO₄. The dried organic phase was then filtered and distillated at vacuum by using a rotary evaporator, so as to remove solvent and obtain a crude product. The crude product was purified by gradient elution through silica gel column chromatography to obtain solid powder S2 (9.5 mol, 95%).

MALDI-TOF MS: C₁₈H₁₂BrN, m/z calculated value: 321.0; measured value: 321.2.

Calculated values from elemental analysis: C, 67.10; H, 3.75; Br, 24.80; N, 4.35; measured values: C, 67.12; H, 3.77; Br, 24.78; N, 4.33.

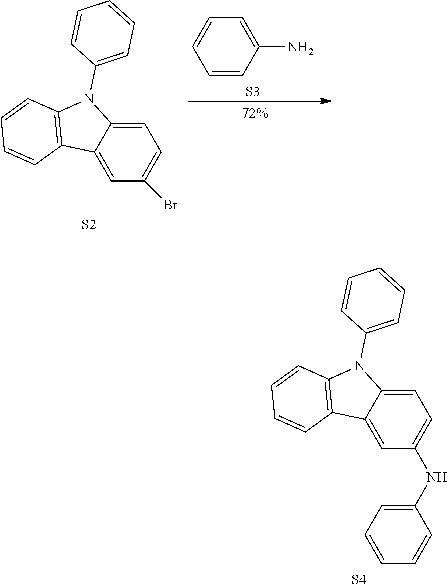

Compound S2 (10 mmol), Compound S3 (10.5 mmol), tris(dibenzylideneacetone) dipalladium (0) (0.05 mmol), sodium tert-butoxide (14 mmol), and tert-butylphosphine (0.2 mmol) were added to a three-necked flask (50 mL). The mixture was degassed for 3 times while stirring, after nitrogen replacement, and 20 mL toluene was added by a syringe. The mixture was heated for reflux for 3 hours under a nitrogen stream, and then cooled to room temperature. After water was added in the cooled reaction solution, the mixture was extracted with dichloromethane, and washed with saturated salt water. The organic phase was dried with anhydrous sodium sulfate. The dried organic phase was then distillated to remove solvent and obtain a crude product. The crude product was purified by column chromatography to obtain an intermediate S4 (7.2 mmol, 72%).

MALDI-TOF MS: C₂₄H₁₈N₂, m/z calculated value: 334.2; measured value: 334.3.

Calculated values from elemental analysis: C, 86.20; H, 5.43; N, 8.38; measured value: C, 86.22; H, 5.41; N, 8.37.

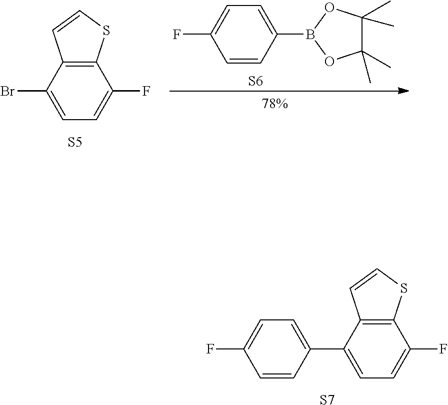

Under the protection of nitrogen, Compound S5 (25 mmol), Compound S6 (25 mmol), [Pd₂(dba)₃].CHCl₃ (0.5 mmol), and HP(tBu)₃.BF₄ (1.0 mmol) were added to a two-necked flask (250 mL). 100 mL toluene (deoxygenated with N₂ for 15 min in advance) was injected into the two-necked flask, then 1M K₂CO₃ water solution (12 mL, deoxygenated with N₂ for 15 min in advance) was added dropwise, and the mixture was stirred overnight at room temperature. After the reaction was completed, 100 mL deionized water and then a few drops of 2 M HCl were added. The mixture was extracted with dichloromethane, and the organic phase was collected and dried with anhydrous Na₂SO₄. The filtered and dried organic phase was distillated by a rotary evaporator to remove solvent, so as to obtain a crude product. The crude product was purified by silica gel chromatography column to obtain a solid S7 (19.5 mmol, 78%).

MALDI-TOF MS: m/z calculated value: C₁₄HaF₂S: 246.0; measured value: 246.3.

Calculated values from elemental analysis: C, 68.28; H, 3.27; F, 15.43; S, 13.02; measured value: C, 68.30; H, 3.29; F, 15.41; S, 13.00.

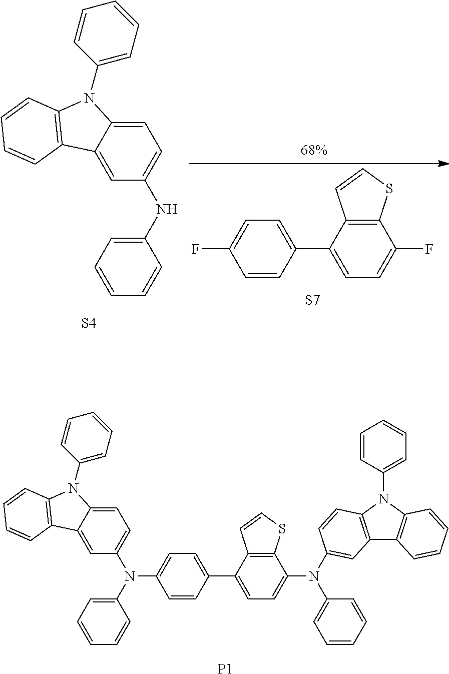 [SMILES: c1ccc(Nc2ccc3c(c2)c2ccccc2n3-c2ccccc2)cc1 ; Fc1ccc(-c2ccc(F)c3sccc23)cc1 ; "S7" ; "S4" ; c1ccc(N(c2ccc(-c3ccc(N(c4ccccc4)c4ccc5c(c4)c4ccccc4n5-c4ccccc4)c4sccc34)cc2)c2ccc3c(c2)c2ccccc2n3-c2ccccc2)cc1 ; "P1"]

Compound S7 (10 mmol/L), Compound S4 (21.5 mmol), tris(dibenzylideneacetone) dipalladium (0) (0.05 mmol), and sodium tert-butoxide (14 mmol), tert-butylphosphine (0.2 mmol) were added to a three-necked flask (50 mL). The mixture was quickly degassed for 3 times while stirring, after carrying out nitrogen replacement, 20 mL toluene was added by a syringe. The mixture was refluxed for 3 hours under a nitrogen stream, and then cooled to room temperature. Water was added to the cooled reaction mixture, extraction was performed with dichloromethane, and the organic phase was washed with saturated salt water. After the organic phase was dried with anhydrous sodium sulfate, solvent in the organic phase was removed by distillation to obtain a crude product. The crude product was purified by column chromatography to obtain Compound P1 (6.8 mmol, 68%).

MALDI-TOF MS: C₆₂H₄₂N₄S, m/z calculated value: 874.3; measured value: 874.5.

Calculated values from elemental analysis: C, 85.10; H, 4.84; N, 6.40; S, 3.66; measured value: C, 85.12; H, 4.86; N, 6.38; S, 3.64.

### Example 2

Synthesis of Compound P4:

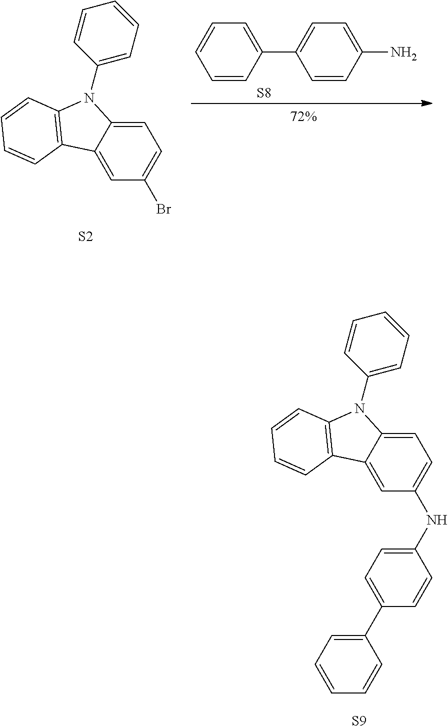

Compound S2 (10 mmol), Compound S8 (10.5 mmol), tris(dibenzylideneacetone) dipalladium (0) (0.05 mmol), sodium tert-butoxide (14 mmol), and tert-butylphosphine (0.2 mmol) were added to a three-necked flask (50 mL). The mixture was quickly degassed for 3 times while stirring, after carrying out nitrogen replacement, 20 mL toluene was added by a syringe. The mixture was refluxed for 3 hours under a nitrogen stream, and then cooled to room temperature. Water was added to the cooled reaction mixture, extraction was performed with dichloromethane, and the organic phase was washed with saturated salt water. After the organic phase was dried with anhydrous sodium sulfate, solvent in the organic phase was removed by distillation and a crude product was obtained. The crude product was purified by column chromatography to obtain an intermediate S9 (7.2 mmol, 72%).

MALDI-TOF MS: C₃₀H₂₂N₂S₄, m/z calculated value: 538.1; measured value: 538.4.

Calculated values from elemental analysis: C, 66.88; H, 4.12; N, 5.20; S, 23.81; measured value: C, 66.90; H, 4.15; N, 5.18; S, 23.78.

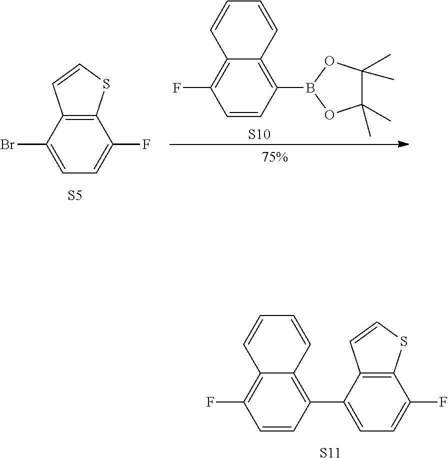

Under protection of nitrogen, Compound S5 (25 mmol), Compound S6 (25 mmol), [Pd₂(dba)₃].CHCl₃ (0.5 mmol), and HP(tBu)₃.BF₄ (1.0 mmol) were weighed and added to a 250 mL two-necked flask. 100 mL toluene (deoxygenated with N₂ for 15 minutes in advance) was injected into the two-necked flask, then 1M K₂CO₃ water solution (12 mL, deoxygenated with N₂ for 15 minutes in advance) was added drop-wise, and the mixture was stirred overnight at room temperature. After the reaction was completed, 100 mL deionized water and then a few drops of 2 M HCl were added. The mixture was extracted with dichloromethane, and the organic phase was collected and dried with anhydrous Na₂SO₄. The filtered and dried organic phase was distillated by a rotary evaporator to remove solvent, so as to obtain a crude product. The crude product was purified by silica gel chromatography column to obtain a solid S7 (19.5 mmol, 78%).

MALDI-TOF MS: C₁₈H₁₀F₂S, m/z calculated value: 296.0; measured value: 296.2.

Calculated values from elemental analysis: C, 72.96; H, 3.40; F, 12.82; S, 10.82; measured value: C, 72.99; H, 3.42; F, 12.80; S, 10.79.

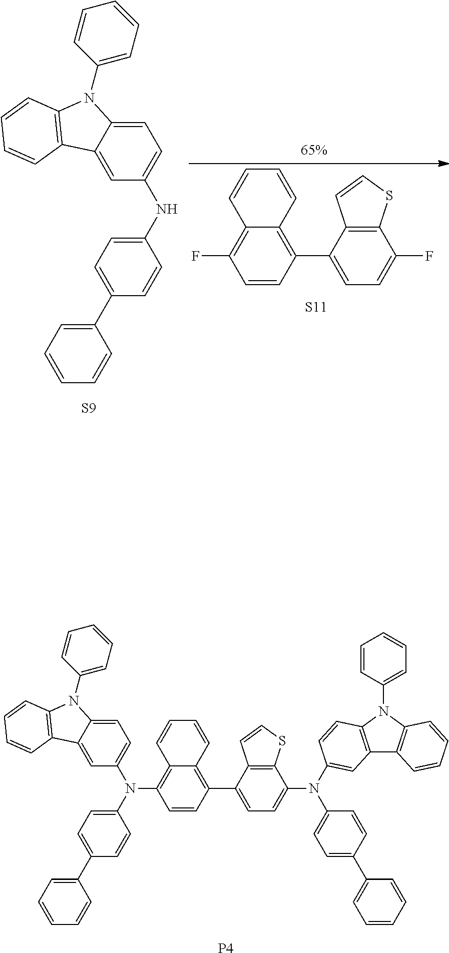

Compound S11 (10 mmol), Compound S9 (20.5 mmol), tris(dibenzylideneacetone) dipalladium (0) (0.05 mmol), sodium tert-butoxide (14 mmol), and tert-butylphosphine (0.2 mmol) were added to a three-necked flask (50 mL). The mixture was quickly degassed for 3 times while stirring, after carrying out nitrogen replacement, 20 mL toluene was added by a syringe. The mixture was refluxed for 3 hours under a nitrogen stream, and then cooled to room temperature. Water was added to the cooled reaction mixture, extraction was performed with dichloromethane, and the organic phase was washed with saturated salt water. After the organic phase was dried with anhydrous sodium sulfate, solvent in the organic phase was removed by distillation to obtain a crude product. The crude product was purified by column chromatography to obtain a Compound P4 (6.5 mmol, 65%).

MALDI-TOF MS: C₇₈H₅₂N₄S, m/z calculated value: 1076.4; measured value: 1076.7.

Calculated values from elemental analysis: C, 86.96; H, 4.87; N, 5.20; S, 2.98; measured value: C, 86.99; H, 4.84; N, 5.21; S, 2.97.

### Example 3

Synthesis of Compound P5:

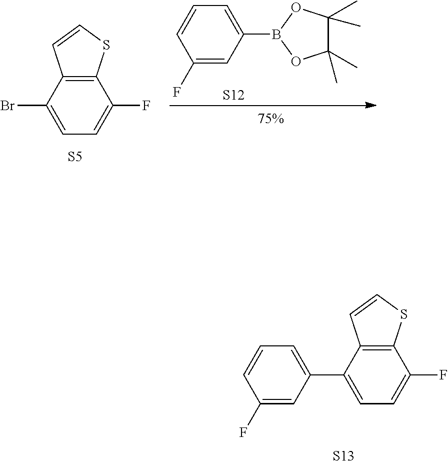

Under protection of nitrogen, Compound S5 (25 mmol), Compound S12 (25 mmol), [Pd₂(dba)₃].CHCl₃ (0.5 mmol), and HP(tBu)₃.BF₄ (1.0 mmol) were weighed and added to a two-necked flask (250 mL). 100 mL toluene (deoxygenated with N₂ for 15 minutes in advance) was injected into the two-necked flask, then 1M K₂CO₃ water solution (12 mL, deoxygenated with N₂ for 15 minutes in advance) was added drop-wise, and the mixture was stirred overnight at room temperature. After the reaction was completed, 100 mL deionized water and then a few drops of 2 M HCl were added. The mixture was extracted with dichloromethane, and the organic phase was collected and dried with anhydrous Na₂SO₄. The filtered and dried organic phase was distillated by a rotary evaporator to remove solvent, so as to obtain a crude product. The crude product was purified by silica gel chromatography column to obtain a solid S13 (18.8 mmol, 75%).

MALDI-TOF MS: C₁₄H₈F₂S, m/z calculated value: 246.0; measured value: 246.4.

Calculated values from elemental analysis: C, 68.28; H, 3.27; F, 15.43; S, 13.02; measured value: C, 68.30; H, 3.25; F, 15.42; S, 13.03.

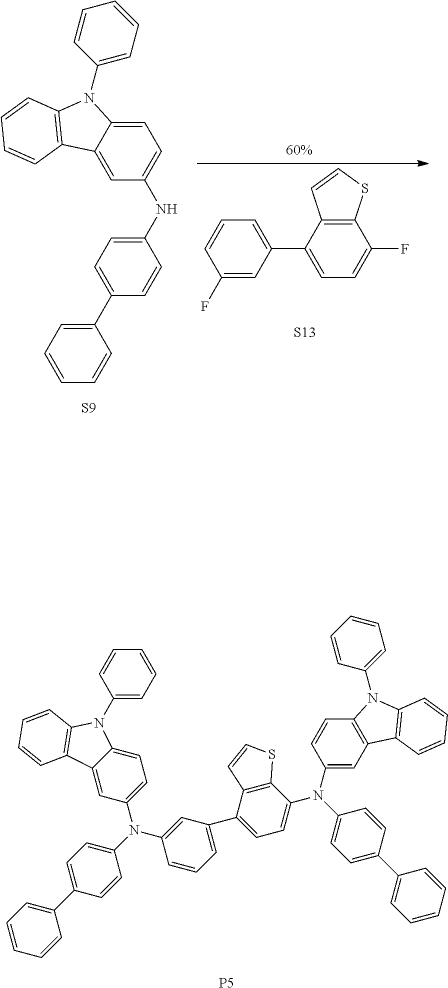

Compound S13 (10 mmol), Compound S9 (21.5 mmol), tris(dibenzylideneacetone) dipalladium (0) (0.05 mmol), sodium tert-butoxide (14 mmol), and tert-butylphosphine (0.2 mmol) were added to a three-necked flask (50 mL). The mixture was quickly degassed for 3 times while stirring, after carrying out nitrogen replacement, 20 mL toluene was added by a syringe. The mixture was refluxed for 3 hours under a nitrogen stream, and then cooled to room temperature. Water was added to the cooled reaction mixture, extraction was performed with dichloromethane, and the organic phase was washed with saturated salt water. After the organic phase was dried with anhydrous sodium sulfate, solvent in the organic phase was removed by distillation to obtain a crude product. The crude product was purified by column chromatography to obtain Compound P5 (6.0 mmol, 60%).

MALDI-TOF MS: C₇₄H₅₀N₄S, m/z calculated value: 1026.4; measured value: 1026.3.

Calculated values from elemental analysis: C, 86.52; H, 4.91; N, 5.45; S, 3.12; measured value: C, 86.55; H, 4.93; N, 5.42; S, 3.10.

### Example 4

Synthesis of Compound P7:

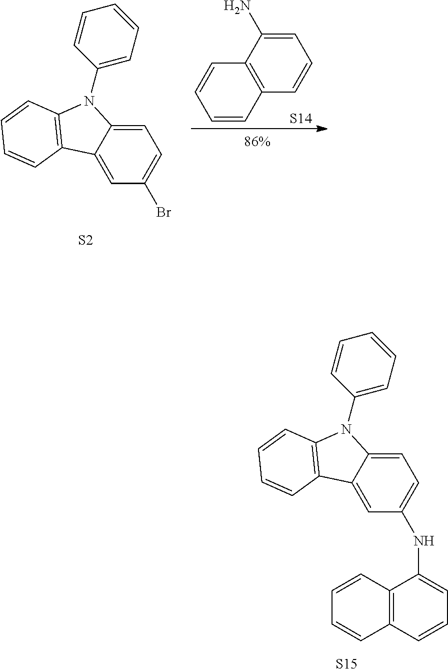

Compound S2 (10 mmol), Compound S14 (10.5 mmol), tris(dibenzylideneacetone) dipalladium (0) (0.05 mmol), sodium tert-butoxide (14 mmol), and tert-butylphosphine (0.2 mmol) were added to a three-necked flask (50 mL). The mixture was quickly degassed for 3 times while stirring, after carrying out nitrogen replacement, 20 mL toluene was added by a syringe. The mixture was refluxed for 3 hours under a nitrogen stream, and then cooled to room temperature. Water was added to the cooled reaction mixture, extraction was performed with dichloromethane, and the organic phase was washed with saturated salt water. After the organic phase was dried with anhydrous sodium sulfate, solvent in the organic phase was removed by distillation to obtain a crude product. The crude product was purified by column chromatography to obtain an intermediate S15 (8.6 mmol, 86%).

MALDI-TOF MS: C₂₈H₂₀N₂, m/z calculated value: 384.2; measured value: 384.5.

Calculated values from elemental analysis: C, 87.47; H, 5.24; N, 7.29; measured value: C, 87.49; H, 5.23; N, 7.28.

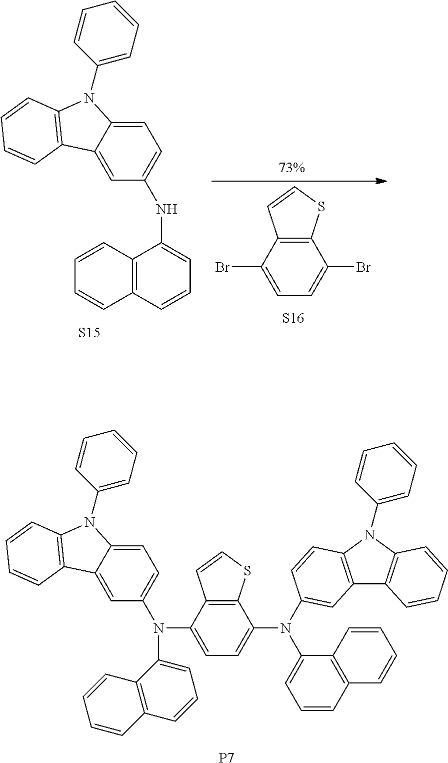

Compound S16 (10 mmol), Compound S15 (21.5 mmol), tris(dibenzylideneacetone) dipalladium (0) (0.05 mmol), sodium tert-butoxide (14 mmol), and tert-butylphosphine (0.2 mmol) were added to a three-necked flask (50 mL). The mixture was quickly degassed for 3 times while stirring, after carrying out nitrogen replacement, 20 mL toluene was added by a syringe. The mixture was refluxed for 3 hours under a nitrogen stream, and then cooled to room temperature. Water was added to the cooled reaction mixture, extraction was performed with dichloromethane, and the organic phase was washed with saturated salt water. After the organic phase was dried with anhydrous sodium sulfate, solvent in the organic phase was removed by distillation to obtain a crude product. The crude product was purified by column chromatography to obtain Compound P7 (7.3 mmol, 73%).

MALDI-TOF MS: C₆₄H₄₂N₄S, m/z calculated value: 898.3; measured value: 898.5.

Calculated values from elemental analysis: C, 85.49; H, 4.71; N, 6.23; S, 3.57; measured value: C, 85.51; H, 4.73; N, 6.21; S, 3.55.

### Example 5

Synthesis of Compound P10:

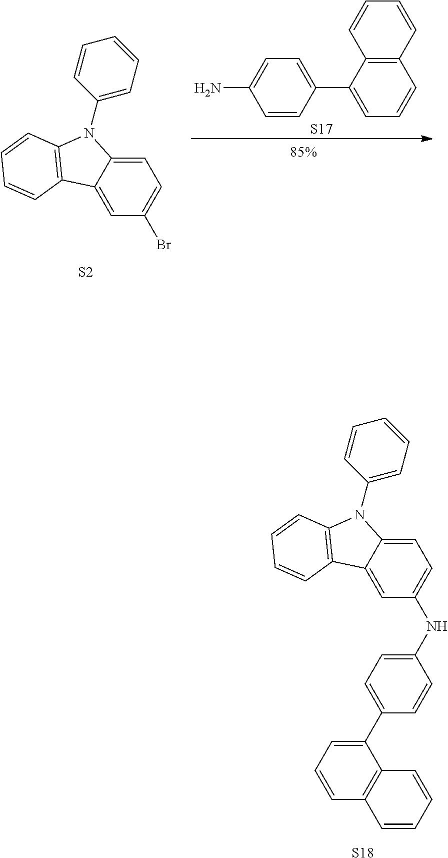

Compound S2 (10 mmol), Compound S17 (10.5 mmol), tris(dibenzylideneacetone) dipalladium (0) (0.05 mmol), sodium tert-butoxide (14 mmol), and tert-butylphosphine (0.2 mmol) were added to a three-necked flask (50 mL). The mixture was quickly degassed for 3 times while stirring, after carrying out nitrogen replacement, 20 mL toluene was added by a syringe. The mixture was refluxed for 3 hours under a nitrogen stream, and then cooled to room temperature. Water was added to the cooled reaction mixture, extraction was performed with dichloromethane, and the organic phase was washed with saturated salt water. After the organic phase was dried with anhydrous sodium sulfate, solvent in the organic phase was removed by distillation to obtain a crude product. The crude product was purified by column chromatography to obtain an intermediate S18 (8.5 mmol, 85%).

MALDI-TOF MS: C₃₄H₂₄N₂, m/z calculated value: 460.2; measured value: 460.6.

Calculated values from elemental analysis: C, 88.67; H, 5.25; N, 6.08; measured value: C, 88.69; H, 5.24; N, 6.07.

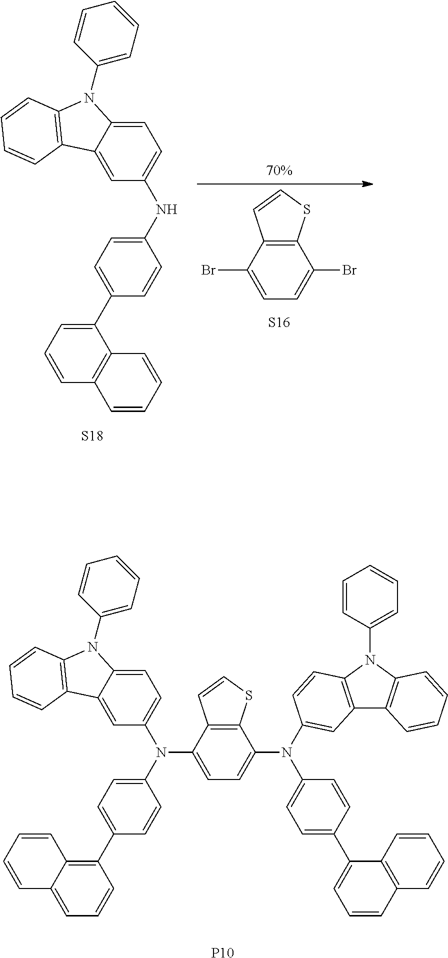

Compound S16 (10 mmol), compound S18 (21.5 mmol), tris(dibenzylideneacetone) dipalladium (0) (0.05 mmol), sodium tert-butoxide (14 mmol), and tert-butylphosphine (0.2 mmol) were added to a three-necked flask (50 mL). The mixture was quickly degassed for 3 times while stirring, after carrying out nitrogen replacement, 20 mL toluene was added by a syringe. The mixture was refluxed for 3 hours under a nitrogen stream, and then cooled to room temperature. Water was added to the cooled reaction mixture, extraction was performed with dichloromethane, and the organic phase was washed with saturated salt water. After the organic phase was dried with anhydrous sodium sulfate, solvent in the organic phase was removed by distillation to obtain a crude product. The crude product was purified by column chromatography to obtain Compound P10 (7.0 mmol, 70%).

MALDI-TOF MS: C₇₆H₅₀N₄S, m/z calculated value: 1050.4; measured value: 1050.6.

Calculated values from elemental analysis: C, 86.83; H, 4.79; N, 5.33; S, 3.05; measured value: C, 86.86; H, 4.81; N, 5.30; S, 3.03.

Table 1 shows respective refractive index (n) and extinction coefficient (k) of several benzo heterocyclic compounds according to the present disclosure and Comparative Compounds Alq3 and CPL1 with respect to light having different wavelengths (450 nm, 550 nm, 630 nm). The Comparative Compounds Alq3 and CPL1 have the following chemical structures.

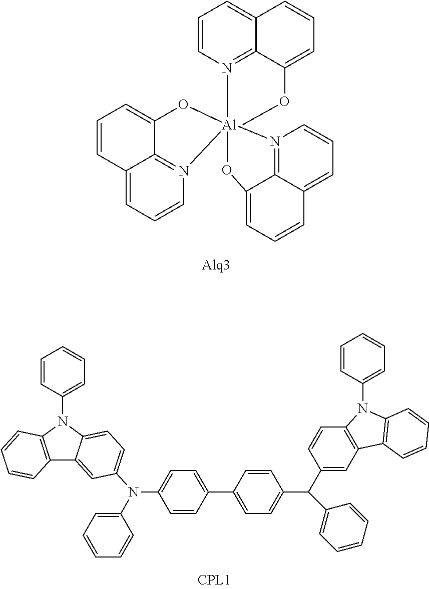

TABLE 1

| | | 450 nm | | 550 nm | | 630 nm | |
|---|---|---|---|---|---|---|---|
| Compound | T_{g}/° C. | n | k | n | k | n | k |
| P1 | 154 | 2.11 | 0.045 | 1.97 | 0.000 | 1.93 | 0.000 |
| P2 | 157 | 2.19 | 0.065 | 2.01 | 0.000 | 1.96 | 0.000 |
| P3 | 163 | 2.25 | 0.085 | 2.08 | 0.000 | 2.03 | 0.000 |
| P4 | 168 | 2.14 | 0.082 | 2.00 | 0.000 | 1.96 | 0.000 |
| P5 | 164 | 2.17 | 0.025 | 2.02 | 0.000 | 1.97 | 0.000 |
| P6 | 160 | 2.12 | 0.052 | 1.97 | 0.000 | 1.93 | 0.000 |
| P7 | 152 | 2.12 | 0.015 | 2.00 | 0.000 | 1.96 | 0.000 |
| P8 | 159 | 2.13 | 0.018 | 2.00 | 0.000 | 1.95 | 0.000 |
| P9 | 158 | 2.19 | 0.028 | 2.05 | 0.000 | 2.00 | 0.000 |
| Alq3 | 149 | 1.78 | 0.156 | 1.73 | 0.000 | 1.71 | 0.000 |
| CPL1 | 150 | 1.96 | 0.001 | 1.83 | 0.000 | 1.78 | 0.000 |

It can be seen from Table 1 that the benzo heterocyclic compounds according to the present disclosure have higher refractive indexes than the Comparative Compounds Alq3 and CPL1, and thus are more conducive to improving the light extraction efficiency. Meanwhile, in view of extinction coefficient, the benzo heterocyclic compounds according to the present disclosure have less absorption in blue light region, and thus are suitable to be used as a light extraction material.

The benzo heterocyclic compounds according to the present disclosure can be used as a material of the capping layer (CPL) of the organic light-emitting component, as well as a material of the auxiliary hole transmission layer and the light-emitting layer.

Example 6 is an embodiment for illustrating technical effects of the nitro benzo heterocyclic compound according to the present disclosure achieved in practical applications.

### Example 6

Organic Light-Emitting Component

The OLED component according to the present embodiment has a structure shown in FIG. 2, in which 1 denotes a substrate, 2 denotes an anode, 3 denotes a hole injection layer, 4 denotes a hole transmission layer, 5 denotes an optical auxiliary layer, and 6 denotes a light-emitting layer, 7 denotes an electron transmission layer, 8 denotes an electron injection layer, 9 denotes a cathode, and 10 denotes a capping layer (CPL).

The substrate 1 and the anode 2 of an ITO film having a film thickness of 100 nm was ultrasonically washed with distilled water, acetone, and isopropanol in sequence, dried in an oven, subjected to UV surface treatment for 30 minutes, and then moved to a vacuum vapor deposition chamber. Respective film layers were then vapor-deposited under a vacuum degree of 2×10⁻⁶ Pa. Compound 1 and Compound 2 (doping weight ratio of 97:3) were vapor-deposited to form the hole injection layer 3 having a thickness of 10 nm, and then Compound 1 was vapor-deposited to form the hole transmission layer (HTL) 4 having a thickness of 100 nm. Compound 3 was vapor-deposited on the hole transmission layer 4 to form the optical auxiliary layer 5 having a thickness of 20 nm. Compound 4, as a doping material of the light-emitting layer 6, and Compound 5, as a host material of the light-emitting layer 6, are vapor-deposited on the optical auxiliary layer 5 at same time, so as to form the light-emitting layer 6 having a thickness of 25 nm. Then Compound 6 and Compound 7 (40:60) were vapor-deposited on the light-emitting layer 6 to form the electron transmission layer (ETL) 7 having a thickness of 50 nm. On the hole transmission layer 7, a LiF layer with a thickness of 2.5 nm and an Ag layer with a thickness of 100 nm were vapor-deposited in sequence, so as to form the electrode injection layer (EIL) 8 and the cathode 9, respectively. Then the capping layer (CPL) 10 having a thickness of 70 nm was formed on a surface on the cathode 9 by vapor deposition. The material of the CPL is selected from a group consisting of the benzo heterocyclic compounds of the present disclosure. In this way, an organic light-emitting component was obtained.

The organic electroluminescent component can also be prepared by a solution processing method.

The solution processing method includes ink-jet printing, spin coating, blade coating, screen printing, roll-to-roll printing, and the like. The solution processing method adopted in the present example was inkjet printing.

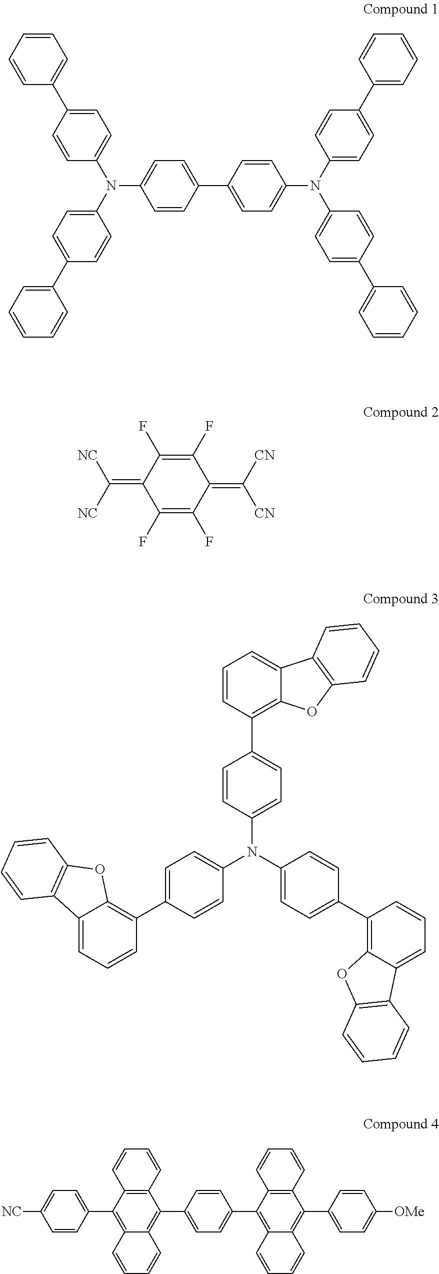 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; N#CC(C#N)=c1c(F)c(F)c(=C(C#N)C#N)c(F)c1F ; c1ccc2c(c1)oc1c(-c3ccc(N(c4ccc(-c5cccc6c5oc5ccccc56)cc4)c4ccc(-c5cccc6c5oc5ccccc56)cc4)cc3)cccc12 ; COc1ccc(-c2c3ccccc3c(-c3ccc(-c4c5ccccc5c(-c5ccc(C#N)cc5)c5ccccc45)cc3)c3ccccc23)cc1]

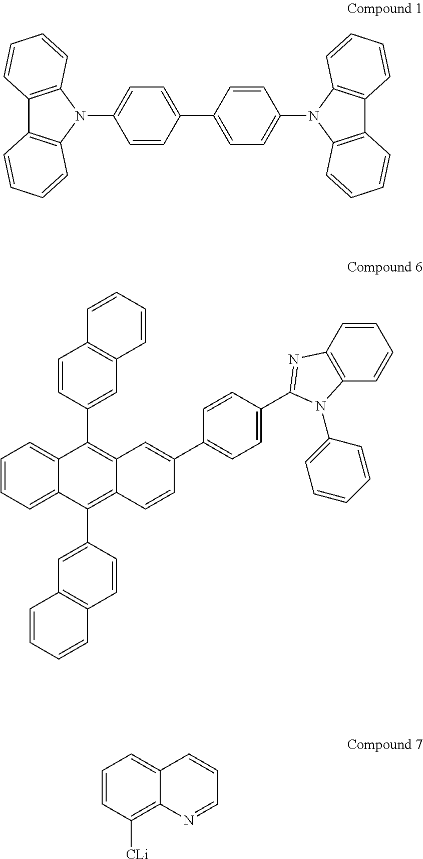

Components 1-10 were prepared in the same manner. In addition, Comparative Components 1-2 were prepared by using Alq3 and CPL1, i.e., the material of CPL in Comparative Component 1 was Alq3, and the material of CPL in Comparative Component 2 was CPL1). In the prepared components merely differ from one another in the material of the CPL, and other layers, such as the light-emitting layer and the auxiliary layer, were the same. These organic light-emitting components were applied with direct current voltage, and their luminescence performances were recorded in Table 2.

TABLE 2
Measurement Results of Luminescence
Performances of Components

| | | | Current | Lifespan |
|---|---|---|---|---|
| | | Driving | Efficiency | (Comparing with |
| | Material | Voltage | (cd/A)@10 | Comparative |
| No. | of CPL | (V) | mA/cm² | Component 2) |
| Component 1 | P1 | 4.02 | 5.22 | 1.13 |
| Component 2 | P2 | 4.15 | 5.32 | 1.08 |
| Component 3 | P3 | 4.14 | 5.42 | 1.10 |
| Component 4 | P4 | 4.13 | 5.28 | 1.12 |
| Component 5 | P5 | 4.10 | 5.38 | 1.20 |
| Component 6 | P6 | 4.04 | 5.25 | 1.06 |
| Component 7 | P7 | 4.08 | 5.18 | 1.14 |
| Component 8 | P8 | 4.16 | 5.16 | 1.15 |
| Component 9 | P9 | 4.13 | 5.45 | 1.10 |
| Comparative | Alq3 | 4.10 | 4.92 | 0.95 |
| Component 1 | | | | |
| Comparative | CPL1 | 4.15 | 4.86 | 1 |
| Component 2 | | | | |

It can be seen from Table 2 that the Components 1-9 using the benzo heterocyclic compounds of the present disclosure have a smaller difference in driving voltage, significantly improved current efficiency, and significantly prolonged lifespan as compared with the Comparative Components 1 and 2 using Alq3 and CPL1. This indicates that the light extraction efficiency can be greatly improved by adopting the benzo heterocyclic compound according to the present disclosure in the capping layer.

In another aspect, the present disclosure provides a display device including the above-mentioned organic light-emitting display panel.

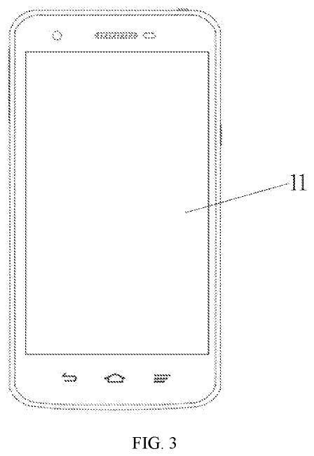

In the present disclosure, the organic light-emitting component may be an organic light-emitting diode (OLED) that can be used in an organic light-emitting display device. The organic light-emitting display device can be display screen of various smart devices, such a mobile phone display screen, a computer display screen, a liquid crystal television display screen, a smart watch display screen, a display panel of smart car, a display screen of Virtual Reality (VR) or Augmented Reality (AR). FIG. 3 is a schematic diagram of a display device according to an embodiment of the present disclosure, in which 11 denotes a mobile phone display screen.

The preferable embodiments of the present disclosure described above are not intended to limit the scope of the present disclosure. Those skilled in the art can make various changes and modifications without departing from the scope of the present disclosure. The protection scope of the present disclosure is defined by the appending claims.

What is claimed is:

1. A benzo heterocyclic compound having a structure represented by Formula (I): 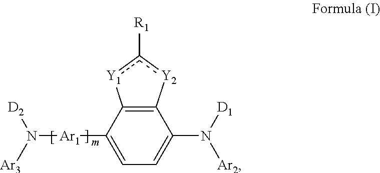
wherein R₁ is selected from a group consisting of hydrogen, C1-C20 alkyl, C1-C20 alkoxy, phenyl, naphthyl, anthracyl, and phenanthryl;
one of Y₁ and Y₂ is carbon, and the other one of Y₁ and Y₂ is oxygen or sulfur;
Ar₁ is selected from a group consisting of a substituted or unsubstituted phenyl, a substituted or unsubstituted naphthyl, a substituted or unsubstituted anthracyl, a substituted or unsubstituted phenanthryl, a substituted or unsubstituted acenaphthylenyl, and a substituted or unsubstituted aromatic heterocyclic group;
m is 0 or 1;
D₁ and D₂ are each independently selected from a group consisting of a substituted or unsubstituted phenyl, a substituted or unsubstituted biphenyl, a substituted or unsubstituted naphthyl, a substituted or unsubstituted anthracyl, a substituted or unsubstituted phenanthryl, a substituted or unsubstituted acenaphthylenyl, a substituted or unsubstituted pyrenyl, a substituted or unsubstituted perylenyl, a substituted or unsubstituted fluorenyl, a substituted or unsubstituted spirodifluorenyl, a substituted or unsubstituted chrysenyl, a substituted or unsubstituted benzophenanthryl, a substituted or unsubstituted benzoanthracyl, a substituted or unsubstituted fluoranthenyl, a substituted or unsubstituted picenyl, a substituted or unsubstituted furyl, a substituted or unsubstituted benzofuryl, a substituted or unsubstituted dibenzofuryl, a substituted or unsubstituted thienyl, a substituted or unsubstituted benzothienyl, a substituted or unsubstituted dibenzothienyl, a substituted or unsubstituted phenoxazinyl, a substituted or unsubstituted phenazinyl, a substituted or unsubstituted phenothiazinyl, a substituted or unsubstituted thioxazinyl, a substituted or unsubstituted thianthrenyl, a carbazole-derived group, an acridine-derived group, diarylamino, a diarylamine-derived group, triarylamino and a triarylamine-derived group; and
Ar₂ and Ar₃ are each independently selected from a group consisting of a substituted or unsubstituted phenyl, a substituted or unsubstituted biphenyl, a substituted or unsubstituted naphthyl, a substituted or unsubstituted anthracyl, a substituted or unsubstituted phenanthryl, a substituted or unsubstituted acenaphthylenyl, a substituted or unsubstituted pyrenyl, a substituted or unsubstituted perylenyl, a substituted or unsubstituted fluorenyl, a substituted or unsubstituted spirodifluorenyl, a substituted or unsubstituted chrysenyl, a substituted or unsubstituted benzophenanthryl, a substituted or unsubstituted benzoanthracyl, a substituted or unsubstituted fluoranthenyl, a substituted or unsubstituted picenyl, a substituted or unsubstituted furyl, a substituted or unsubstituted benzofuryl, a substituted or unsubstituted dibenzofuryl, a substituted or unsubstituted thienyl, a substituted or unsubstituted benzothienyl, a substituted or unsubstituted dibenzothienyl, a substituted or unsubstituted phenoxazinyl, a substituted or unsubstituted phenazinyl, a substituted or unsubstituted phenothiazinyl, a substituted or unsubstituted thioxazinyl, a substituted or unsubstituted thianthrenyl, a carbazole-derived group, an acridine-derived group, diarylamino, a diarylamine-derived group, triarylamino and a triarylamine-derived group.

2. The benzo heterocyclic compound according to claim 1, having a structure represented by Formula (I-1):

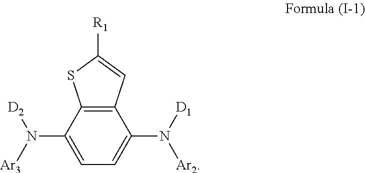

3. The benzo heterocyclic compound according to claim 1, having a structure represented by Formula (I-2):

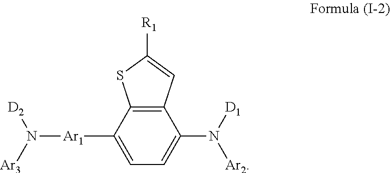

4. The benzo heterocyclic compound according to claim 1, having a structure represented by Formula (I-3):

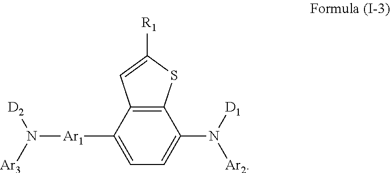

5. The benzo heterocyclic compound according to claim 1, wherein the aromatic heterocyclic group is selected from a group consisting of thienyl, thiazolyl, thiadiazolyl, furyl, oxazolyl, and oxadiazolyl.

6. The benzo heterocyclic compound according to claim 1, wherein D₁ and D₂ are each independently selected from a group consisting of a carbazole-derived group, an acridine-derived group, diarylamino, a diarylamine-derived group, triarylamino and a triarylamine-derived group, and
Ar₂ and Ar₃ are each independently selected from a group consisting of a substituted or unsubstituted phenyl, a substituted or unsubstituted biphenyl, a substituted or unsubstituted naphthyl, and a substituted or unsubstituted anthracyl.

7. The benzo heterocyclic compound according to claim 1, wherein the carbazole-derived group is selected from a group consisting of the following groups: 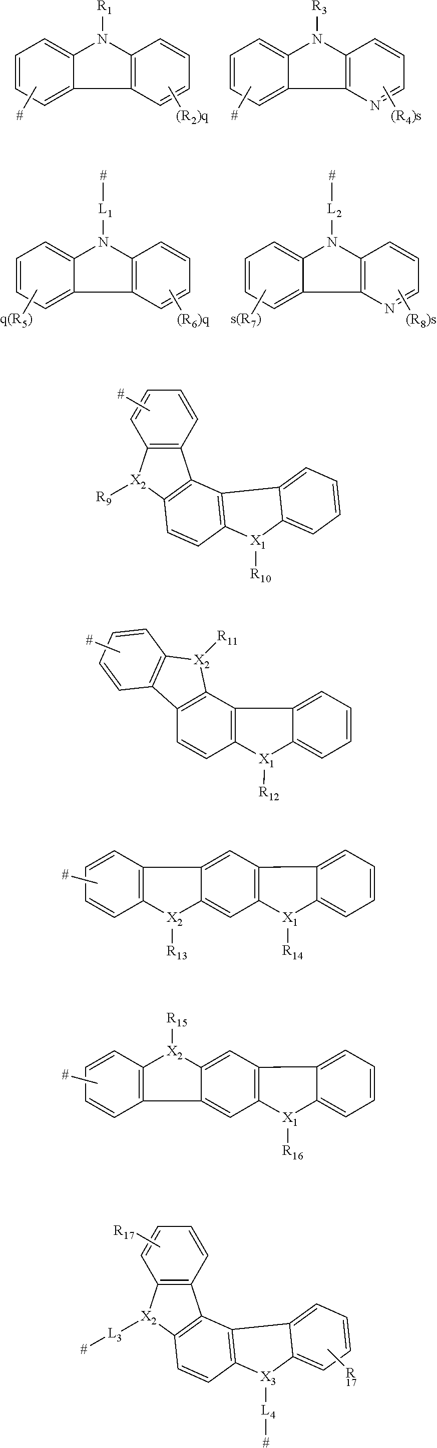 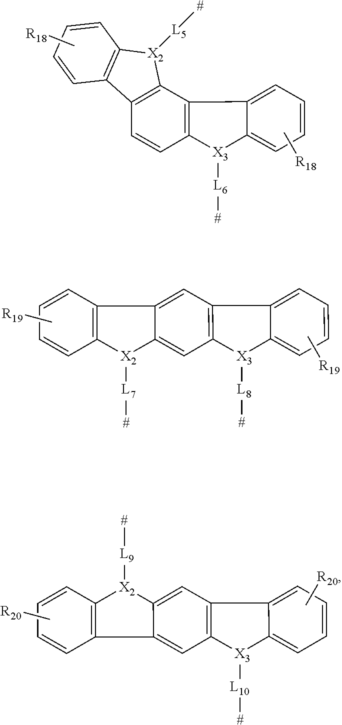
wherein X₁, X₂ and X₃ are each independently selected from a group consisting of carbon, nitrogen, oxygen, sulfur, and silicon, and at least one of X₁, X₂ and X₃ is nitrogen;
R₁-R₂₀ are each independently selected from a group consisting of alkyl, alkoxy, an aromatic ring group, an aromatic fused ring group, and an aromatic heterocyclic group;
q and s are integers independently selected from 0, 1, 2, and 3;
L₁-L₁₀ are each independently selected from a group consisting of an aromatic ring group, an aromatic fused ring group, and an aromatic heterocyclic group; and
# represents a bonding position.

8. The benzo heterocyclic compound according to claim 7, wherein the aromatic ring group is phenyl,
the aromatic fused ring group is selected from a group consisting of naphthyl, anthracyl, pyrenyl, and perylenyl, and
the aromatic heterocyclic group is selected from a group consisting of thienyl, furyl, and thiazolyl.

9. The benzo heterocyclic compound according to claim 7, wherein the carbazole-derived group is selected from a group consisting of the following groups:

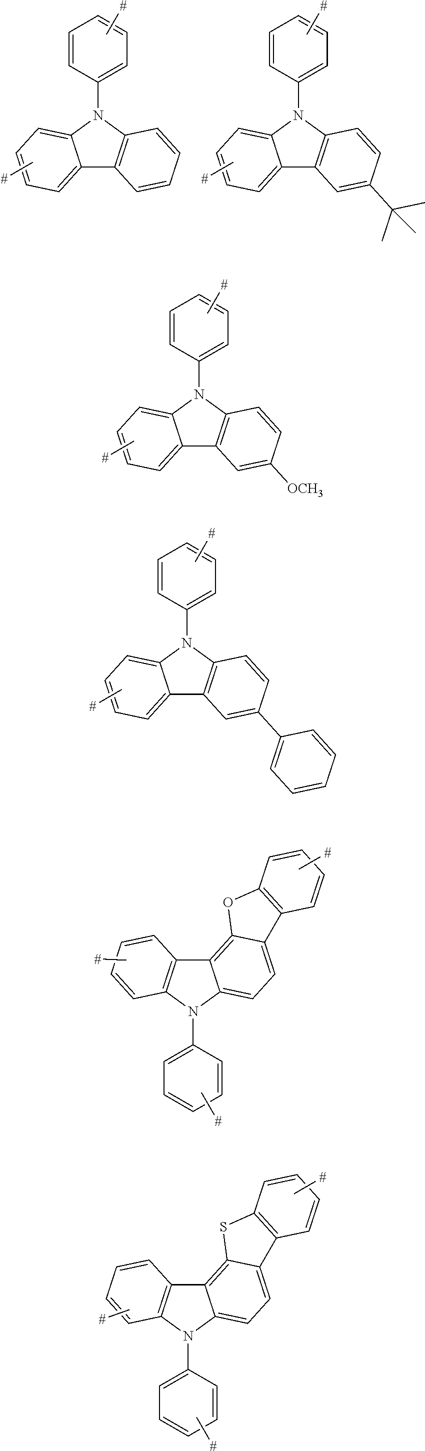

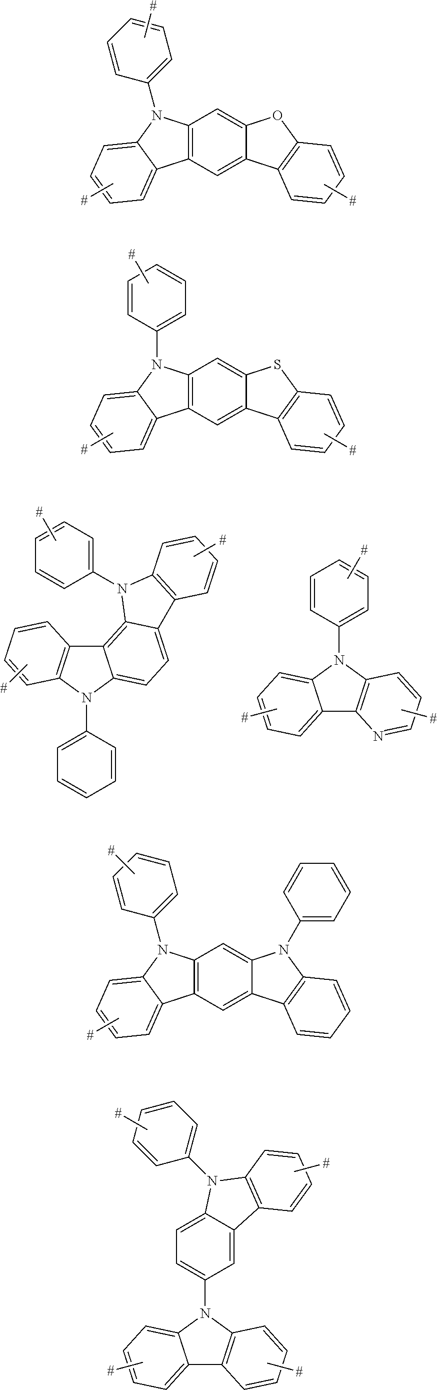

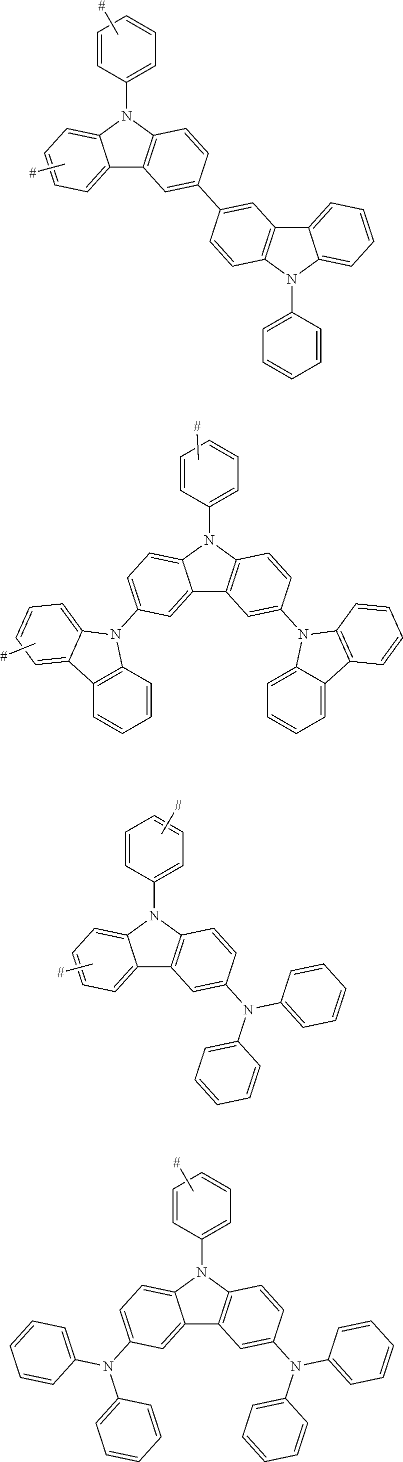

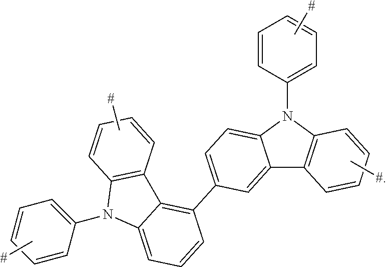

10. The benzo heterocyclic compound according to claim 1, wherein the acridine-derived group is selected from a group consisting of the following groups: 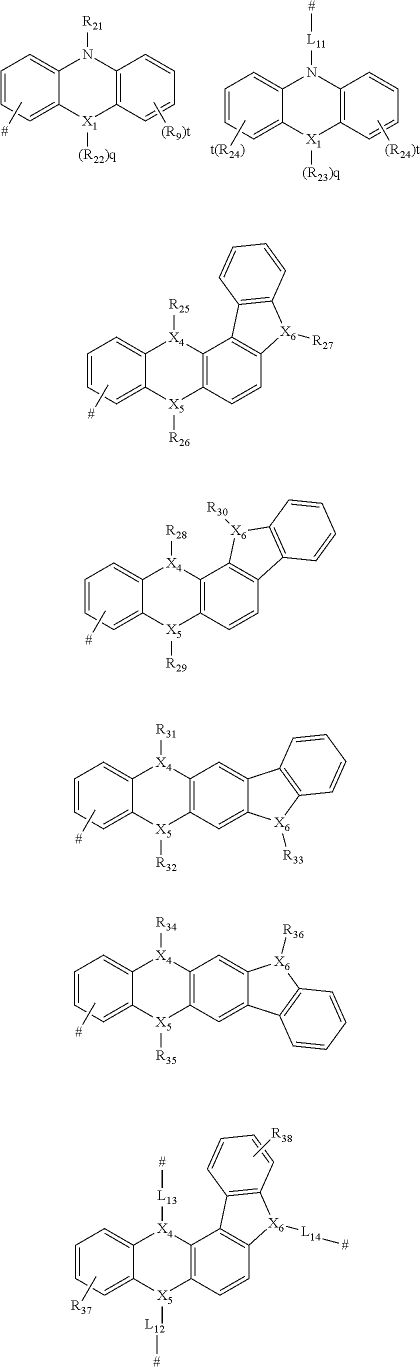 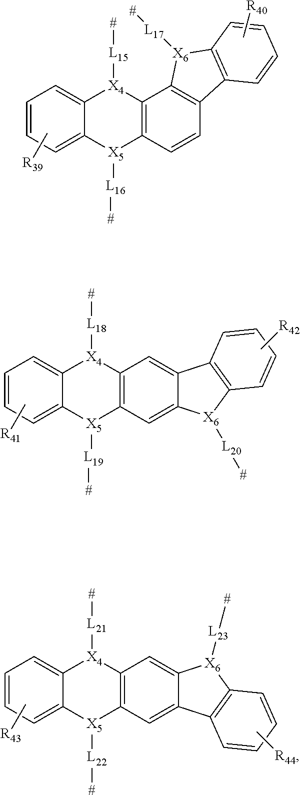
wherein X₁, X₄, X₅, and X₆ are each independently selected from a group consisting of carbon, nitrogen, oxygen, sulfur, and silicon, and at least one of X₄ and X₅ is nitrogen;
R₂₁-R₄₄ are each independently selected from a group consisting of alkyl, alkoxy, an aromatic ring group, an aromatic fused ring group, and an aromatic heterocyclic group, q and t are integers independently selected from 0, 1, 2, and 3;
L₁₁-L₂₃ are each independently selected from a group consisting of an aromatic ring group, an aromatic fused ring group, and an aromatic heterocyclic group; and
# represents a bonding position.

11. The benzo heterocyclic compound according to claim 10, wherein the acridine-derived group is selected from a group consisting of the following groups:

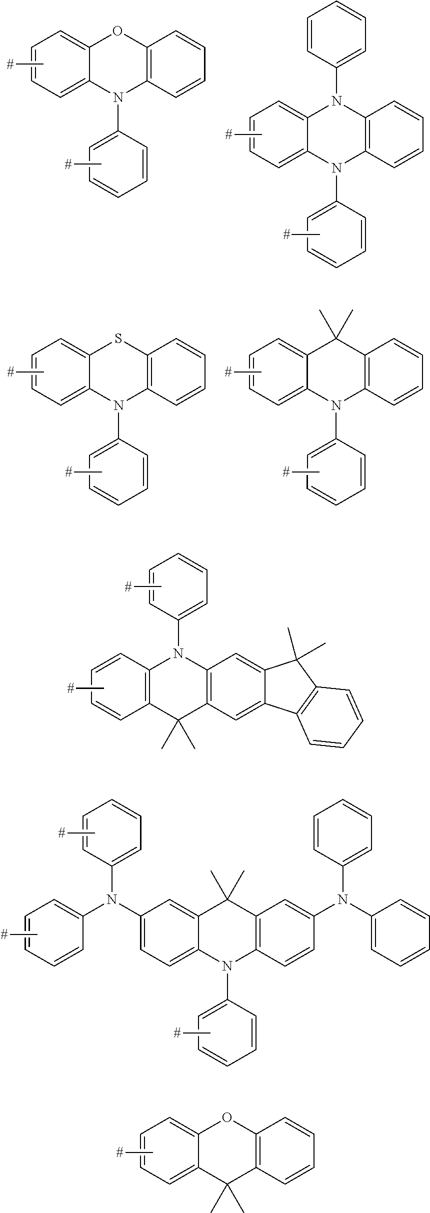

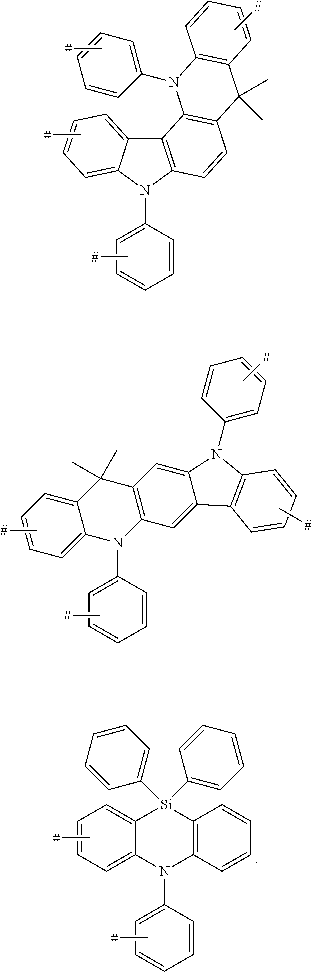

12. The benzo heterocyclic compound according to claim 1, wherein the diarylamine group or the triarylamine group is selected from a group consisting of the following groups: 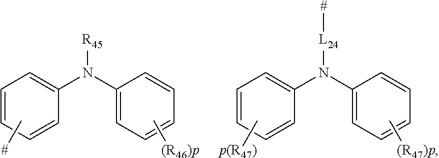
wherein R₄₅-R₄₇ are each independently selected from a group consisting of alkyl, alkoxy, an aromatic ring group, an aromatic fused ring group, and an aromatic heterocyclic group; p is an integer selected from 0, 1, 2, and 3;
L₂₄ is selected from a group consisting of an aromatic ring group, an aromatic fused ring group, and an aromatic heterocyclic group; and
# represents a bonding position.

13. The benzo heterocyclic compound according to claim 12, wherein the diarylamine group or the triarylamine group is selected from a group consisting of the following groups:

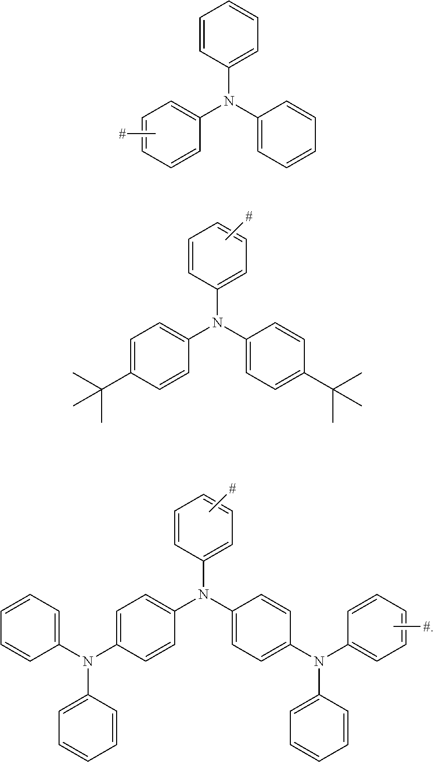

14. The benzo heterocyclic compound according to claim 1, wherein D₁ and D₂ are each independently selected from a group consisting of the following groups: 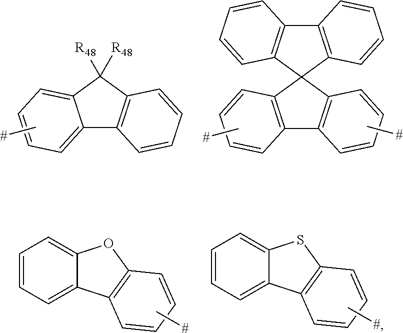
wherein R₄₈ is selected from a group consisting of alkyl, alkoxy, an aromatic ring group, an aromatic fused ring group, and an aromatic heterocyclic group.

15. The benzo heterocyclic compound according to claim 1, wherein the benzo heterocyclic compound is selected from a group consisting of the following compounds:

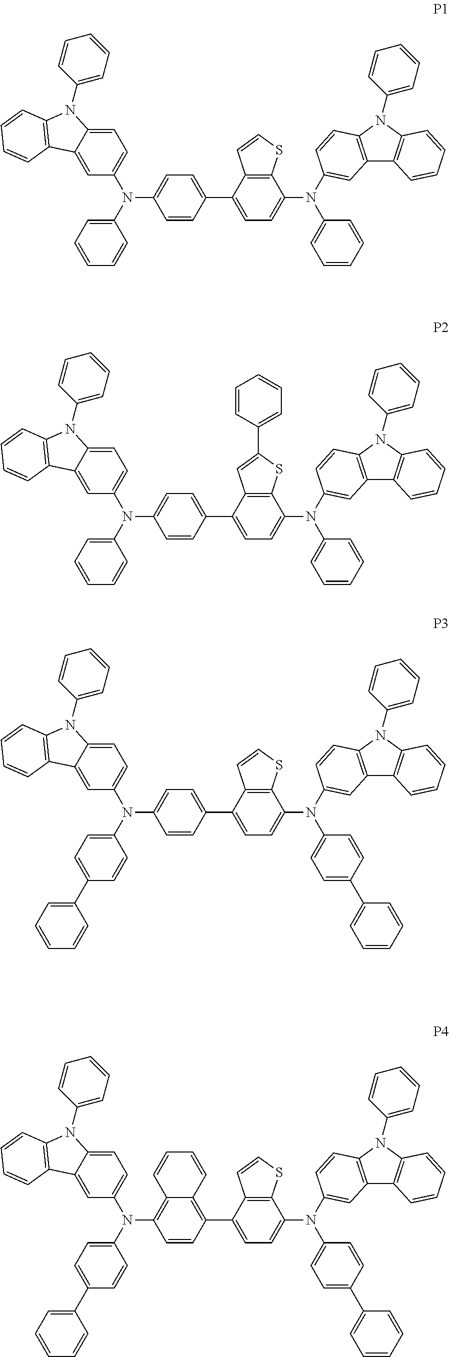

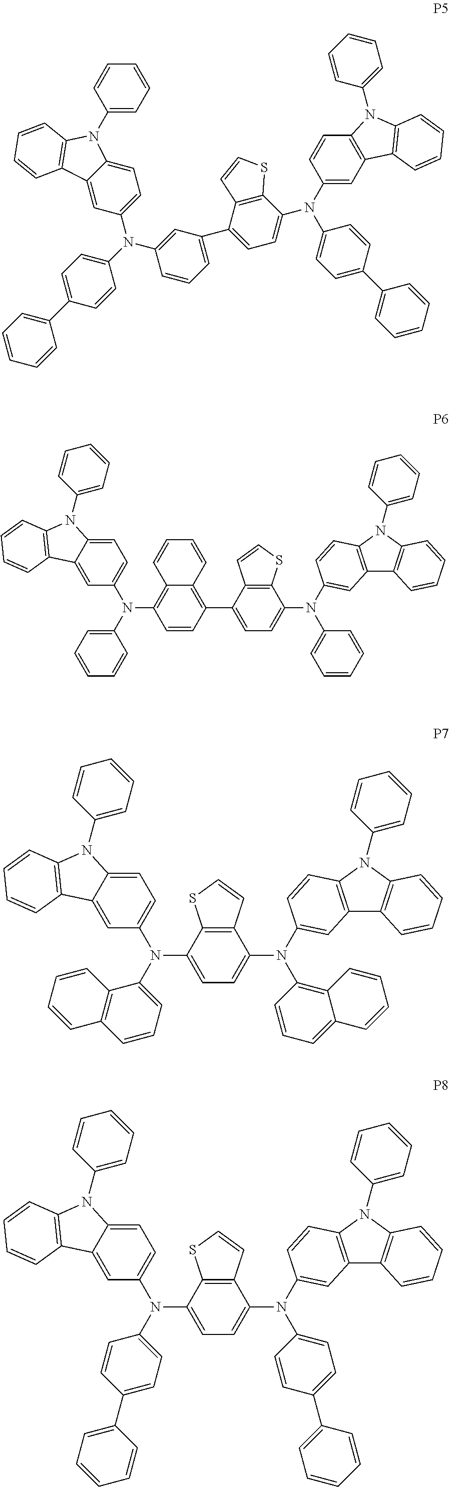 [SMILES: c1ccc(-c2ccc(N(c3cccc(-c4ccc(N(c5ccc(-c6ccccc6)cc5)c5ccc6c(c5)c5ccccc5n6-c5ccccc5)c5sccc45)c3)c3ccc4c(c3)c3ccccc3n4-c3ccccc3)cc2)cc1 ; c1ccc(N(c2ccc3c(c2)c2ccccc2n3-c2ccccc2)c2ccc(-c3ccc(N(c4ccccc4)c4ccc5c(c4)c4ccccc4n5-c4ccccc4)c4sccc34)c3ccccc23)cc1 ; c1ccc(-n2c3ccccc3c3cc(N(c4cccc5ccccc45)c4ccc(N(c5ccc6c(c5)c5ccccc5n6-c5ccccc5)c5cccc6ccccc56)c5sccc45)ccc32)cc1 ; c1ccc(-c2ccc(N(c3ccc4c(c3)c3ccccc3n4-c3ccccc3)c3ccc(N(c4ccc(-c5ccccc5)cc4)c4ccc5c(c4)c4ccccc4n5-c4ccccc4)c4sccc34)cc2)cc1]

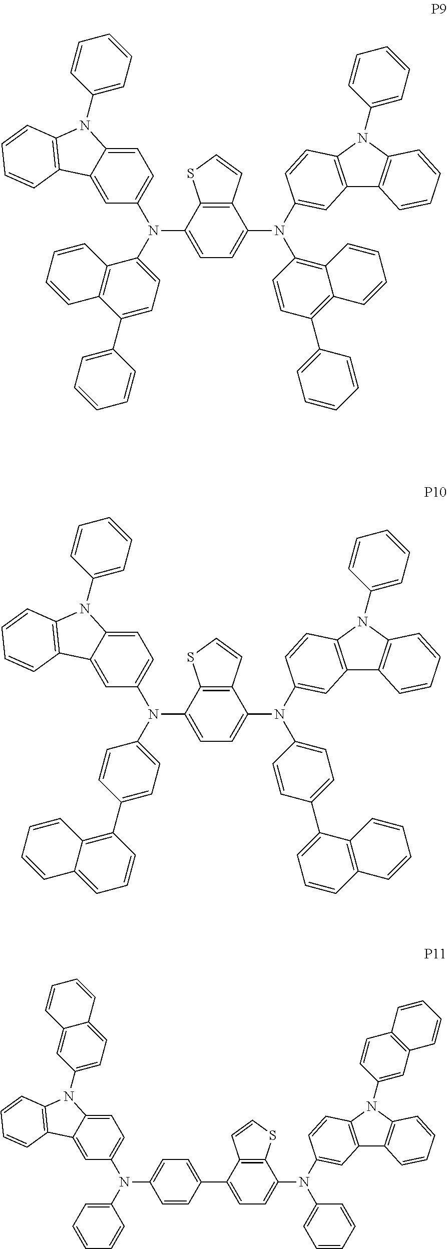 [SMILES: c1ccc(-c2ccc(N(c3ccc4c(c3)c3ccccc3n4-c3ccccc3)c3ccc(N(c4ccc5c(c4)c4ccccc4n5-c4ccccc4)c4ccc(-c5ccccc5)c5ccccc45)c4sccc34)c3ccccc23)cc1 ; c1ccc(-n2c3ccccc3c3cc(N(c4ccc(-c5cccc6ccccc56)cc4)c4ccc(N(c5ccc(-c6cccc7ccccc67)cc5)c5ccc6c(c5)c5ccccc5n6-c5ccccc5)c5sccc45)ccc32)cc1 ; c1ccc(N(c2ccc(-c3ccc(N(c4ccccc4)c4ccc5c(c4)c4ccccc4n5-c4ccc5ccccc5c4)c4sccc34)cc2)c2ccc3c(c2)c2ccccc2n3-c2ccc3ccccc3c2)cc1]

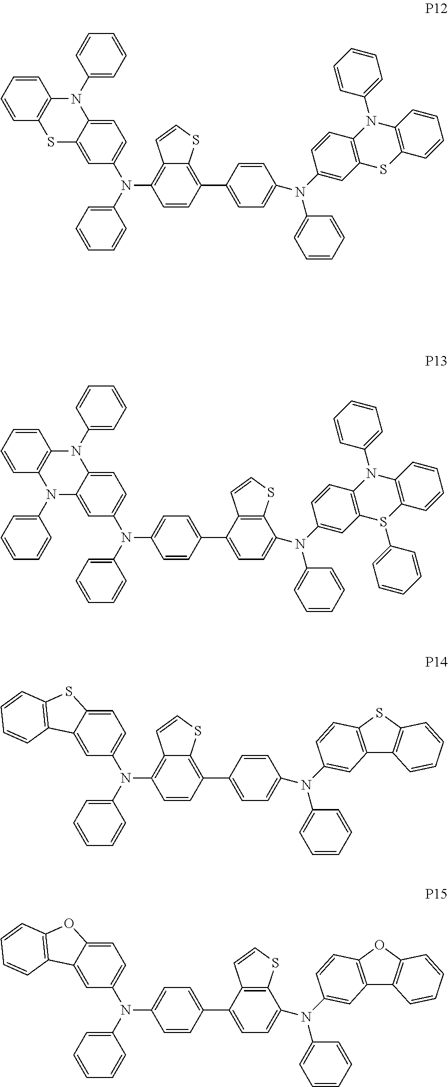

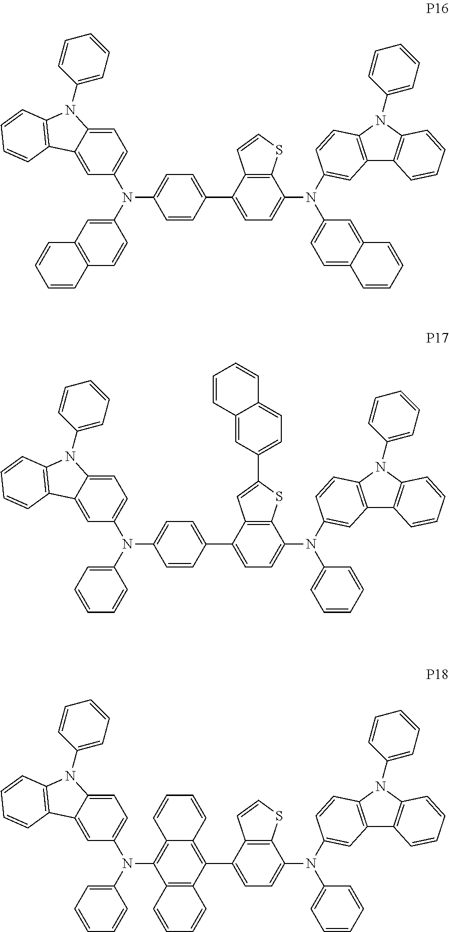 [SMILES: c1ccc(-n2c3ccccc3c3cc(N(c4ccc(-c5ccc(N(c6ccc7ccccc7c6)c6ccc7c(c6)c6ccccc6n7-c6ccccc6)c6sccc56)cc4)c4ccc5ccccc5c4)ccc32)cc1 ; c1ccc(N(c2ccc(-c3ccc(N(c4ccccc4)c4ccc5c(c4)c4ccccc4n5-c4ccccc4)c4sc(-c5ccc6ccccc6c5)cc34)cc2)c2ccc3c(c2)c2ccccc2n3-c2ccccc2)cc1 ; c1ccc(N(c2ccc3c(c2)c2ccccc2n3-c2ccccc2)c2c3ccccc3c(-c3ccc(N(c4ccccc4)c4ccc5c(c4)c4ccccc4n5-c4ccccc4)c4sccc34)c3ccccc23)cc1]

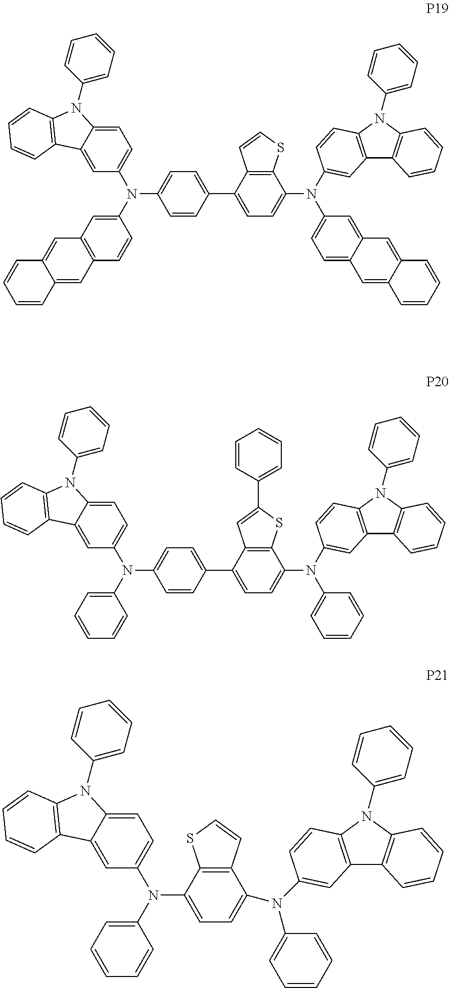 [SMILES: c1ccc(-n2c3ccccc3c3cc(N(c4ccc(-c5ccc(N(c6ccc7cc8ccccc8cc7c6)c6ccc7c(c6)c6ccccc6n7-c6ccccc6)c6sccc56)cc4)c4ccc5cc6ccccc6cc5c4)ccc32)cc1 ; c1ccc(-c2cc3c(-c4ccc(N(c5ccccc5)c5ccc6c(c5)c5ccccc5n6-c5ccccc5)cc4)ccc(N(c4ccccc4)c4ccc5c(c4)c4ccccc4n5-c4ccccc4)c3s2)cc1 ; c1ccc(N(c2ccc3c(c2)c2ccccc2n3-c2ccccc2)c2ccc(N(c3ccccc3)c3ccc4c(c3)c3ccccc3n4-c3ccccc3)c3sccc23)cc1]

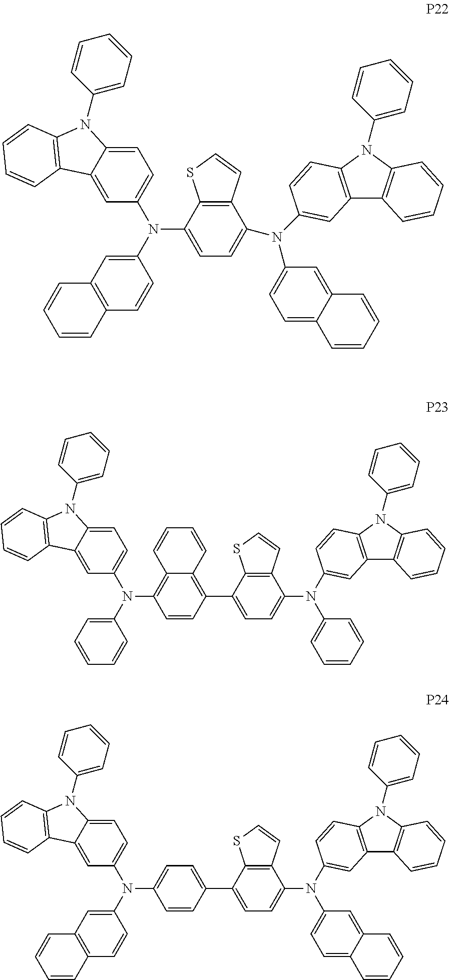 [SMILES: c1ccc(-n2c3ccccc3c3cc(N(c4ccc5ccccc5c4)c4ccc(N(c5ccc6ccccc6c5)c5ccc6c(c5)c5ccccc5n6-c5ccccc5)c5sccc45)ccc32)cc1 ; c1ccc(N(c2ccc3c(c2)c2ccccc2n3-c2ccccc2)c2ccc(-c3ccc(N(c4ccccc4)c4ccc5c(c4)c4ccccc4n5-c4ccccc4)c4ccsc34)c3ccccc23)cc1 ; c1ccc(-n2c3ccccc3c3cc(N(c4ccc(-c5ccc(N(c6ccc7ccccc7c6)c6ccc7c(c6)c6ccccc6n7-c6ccccc6)c6ccsc56)cc4)c4ccc5ccccc5c4)ccc32)cc1]

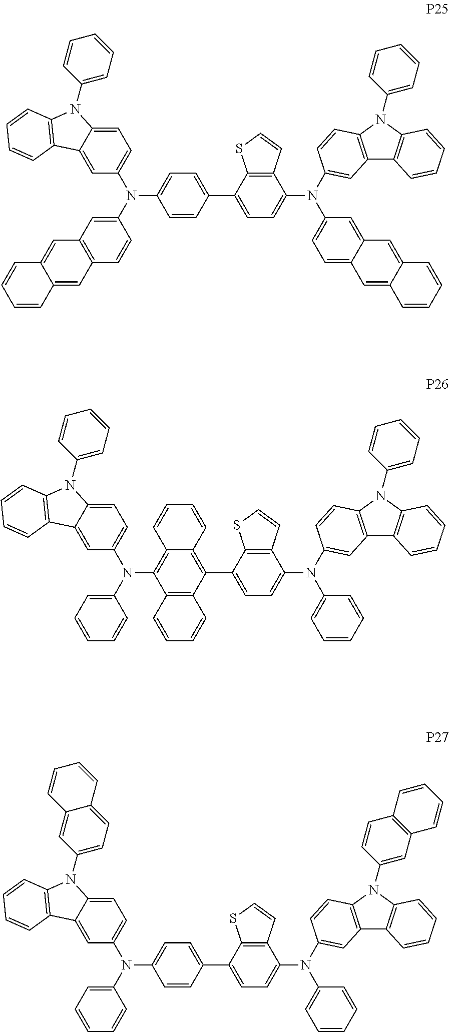 [SMILES: c1ccc(-n2c3ccccc3c3cc(N(c4ccc(-c5ccc(N(c6ccc7cc8ccccc8cc7c6)c6ccc7c(c6)c6ccccc6n7-c6ccccc6)c6ccsc56)cc4)c4ccc5cc6ccccc6cc5c4)ccc32)cc1 ; c1ccc(N(c2ccc3c(c2)c2ccccc2n3-c2ccccc2)c2c3ccccc3c(-c3ccc(N(c4ccccc4)c4ccc5c(c4)c4ccccc4n5-c4ccccc4)c4ccsc34)c3ccccc23)cc1 ; c1ccc(N(c2ccc(-c3ccc(N(c4ccccc4)c4ccc5c(c4)c4ccccc4n5-c4ccc5ccccc5c4)c4ccsc34)cc2)c2ccc3c(c2)c2ccccc2n3-c2ccc3ccccc3c2)cc1]

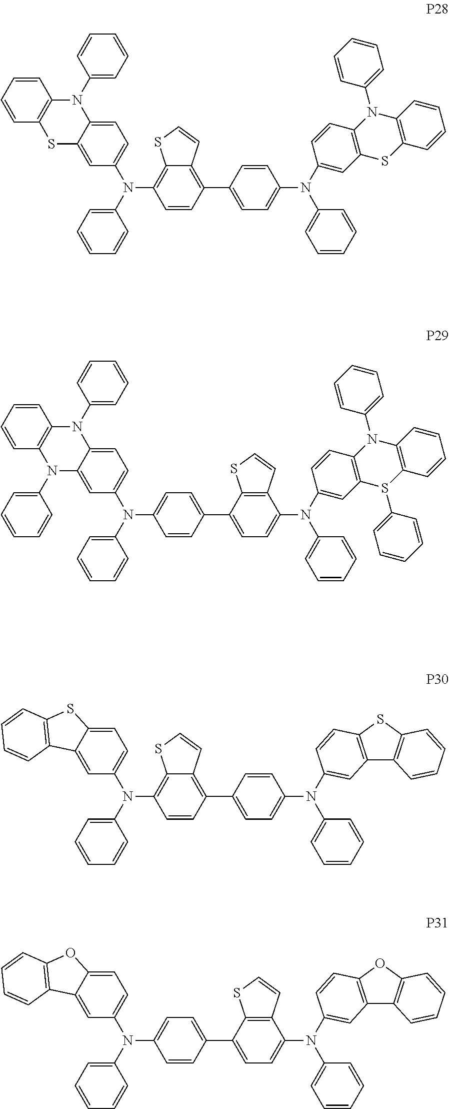

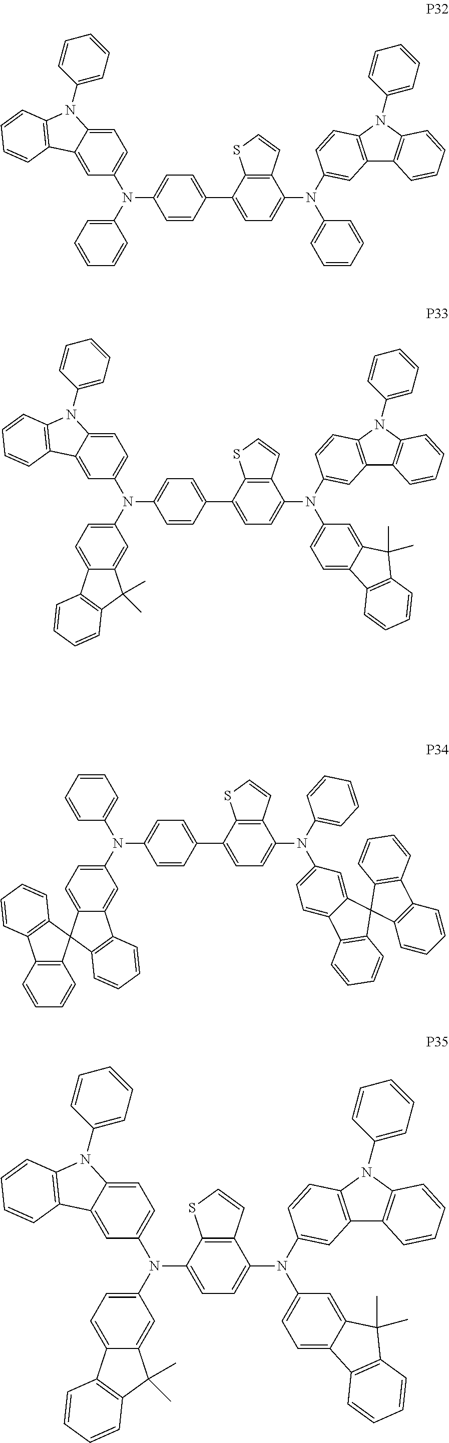

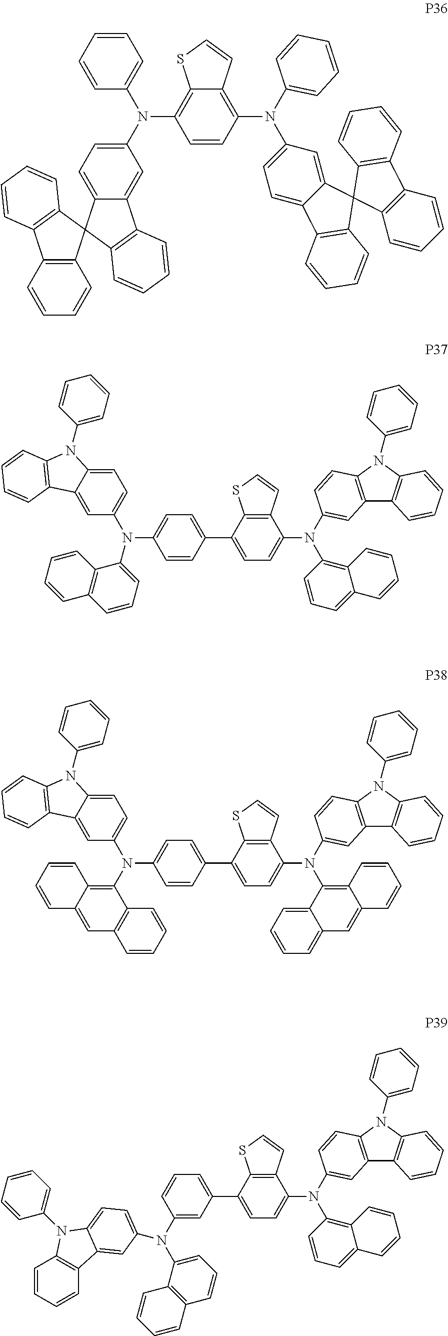

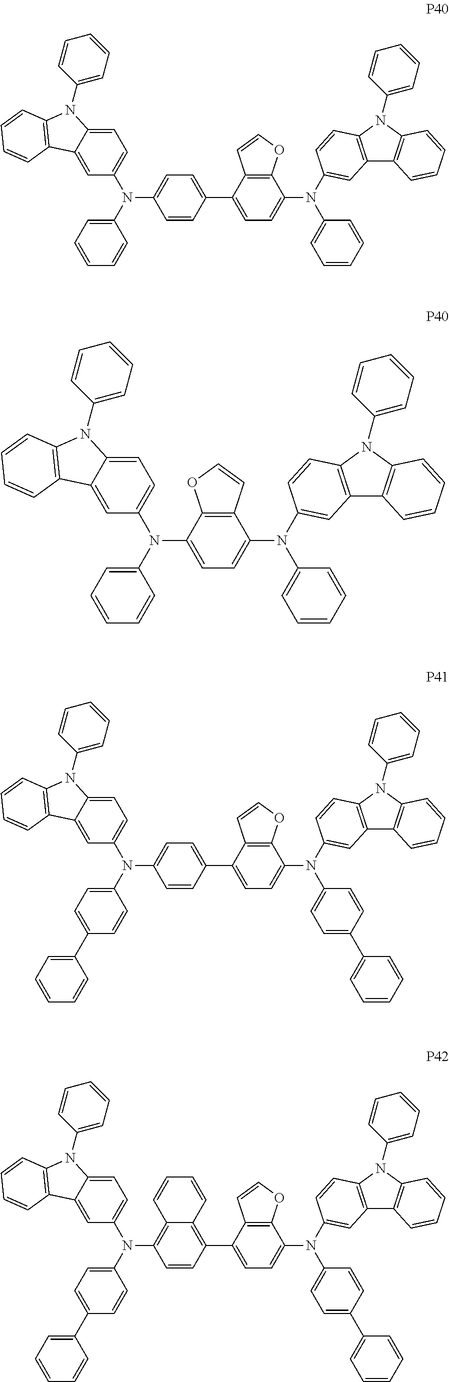 [SMILES: c1ccc(N(c2ccc(-c3ccc(N(c4ccccc4)c4ccc5c(c4)c4ccccc4n5-c4ccccc4)c4occc34)cc2)c2ccc3c(c2)c2ccccc2n3-c2ccccc2)cc1 ; c1ccc(N(c2ccc3c(c2)c2ccccc2n3-c2ccccc2)c2ccc(N(c3ccccc3)c3ccc4c(c3)c3ccccc3n4-c3ccccc3)c3occc23)cc1 ; c1ccc(-c2ccc(N(c3ccc(-c4ccc(N(c5ccc(-c6ccccc6)cc5)c5ccc6c(c5)c5ccccc5n6-c5ccccc5)c5occc45)cc3)c3ccc4c(c3)c3ccccc3n4-c3ccccc3)cc2)cc1 ; c1ccc(-c2ccc(N(c3ccc4c(c3)c3ccccc3n4-c3ccccc3)c3ccc(-c4ccc(N(c5ccc(-c6ccccc6)cc5)c5ccc6c(c5)c5ccccc5n6-c5ccccc5)c5occc45)c4ccccc34)cc2)cc1]

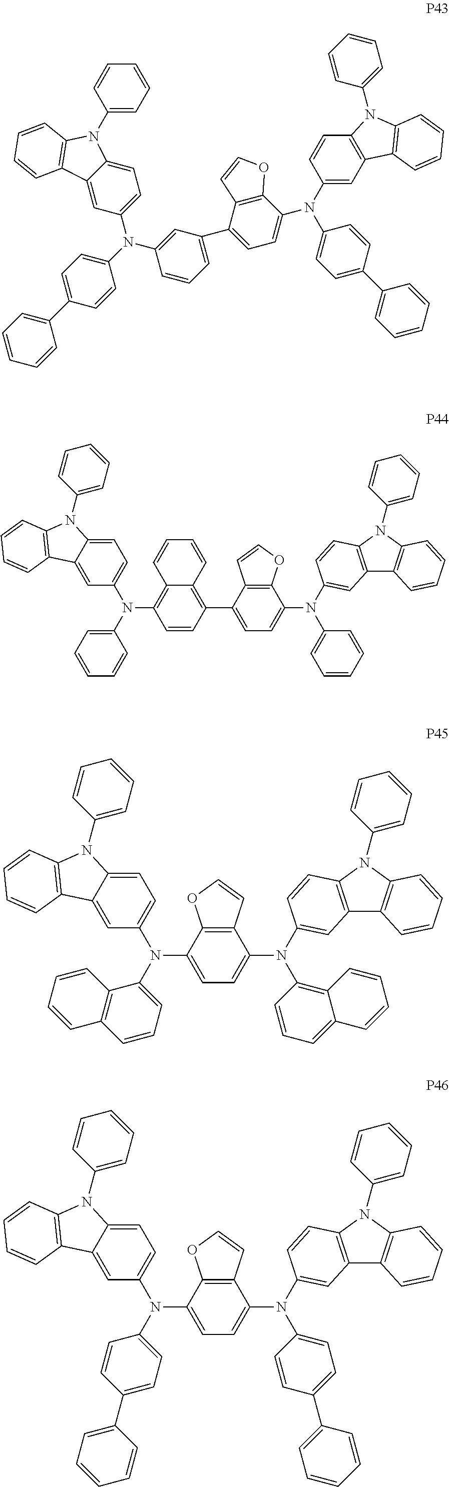 [SMILES: c1ccc(-c2ccc(N(c3cccc(-c4ccc(N(c5ccc(-c6ccccc6)cc5)c5ccc6c(c5)c5ccccc5n6-c5ccccc5)c5occc45)c3)c3ccc4c(c3)c3ccccc3n4-c3ccccc3)cc2)cc1 ; c1ccc(N(c2ccc3c(c2)c2ccccc2n3-c2ccccc2)c2ccc(-c3ccc(N(c4ccccc4)c4ccc5c(c4)c4ccccc4n5-c4ccccc4)c4occc34)c3ccccc23)cc1 ; c1ccc(-n2c3ccccc3c3cc(N(c4cccc5ccccc45)c4ccc(N(c5ccc6c(c5)c5ccccc5n6-c5ccccc5)c5cccc6ccccc56)c5occc45)ccc32)cc1 ; c1ccc(-c2ccc(N(c3ccc4c(c3)c3ccccc3n4-c3ccccc3)c3ccc(N(c4ccc(-c5ccccc5)cc4)c4ccc5c(c4)c4ccccc4n5-c4ccccc4)c4occc34)cc2)cc1]

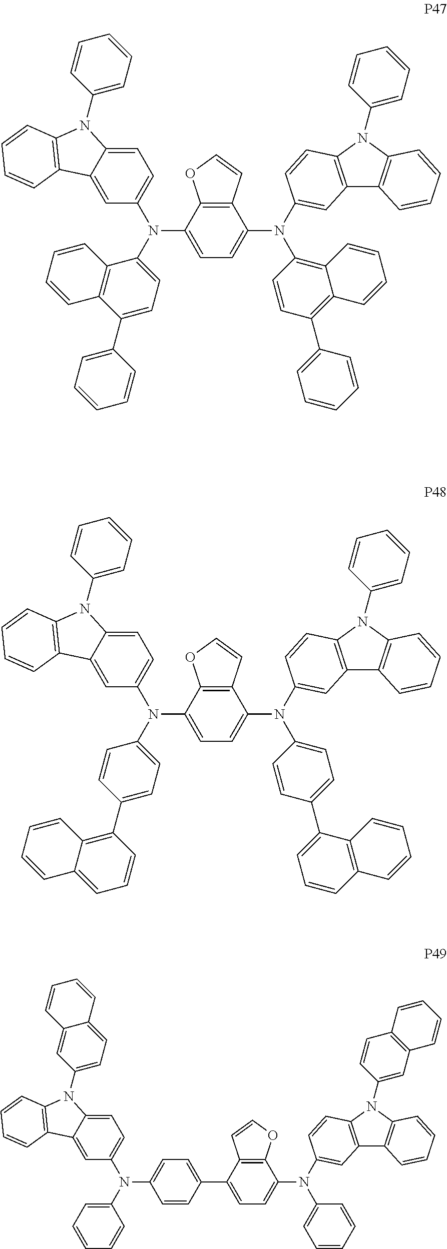 [SMILES: c1ccc(-c2ccc(N(c3ccc4c(c3)c3ccccc3n4-c3ccccc3)c3ccc(N(c4ccc5c(c4)c4ccccc4n5-c4ccccc4)c4ccc(-c5ccccc5)c5ccccc45)c4occc34)c3ccccc23)cc1 ; c1ccc(-n2c3ccccc3c3cc(N(c4ccc(-c5cccc6ccccc56)cc4)c4ccc(N(c5ccc(-c6cccc7ccccc67)cc5)c5ccc6c(c5)c5ccccc5n6-c5ccccc5)c5occc45)ccc32)cc1 ; c1ccc(N(c2ccc(-c3ccc(N(c4ccccc4)c4ccc5c(c4)c4ccccc4n5-c4ccc5ccccc5c4)c4occc34)cc2)c2ccc3c(c2)c2ccccc2n3-c2ccc3ccccc3c2)cc1]

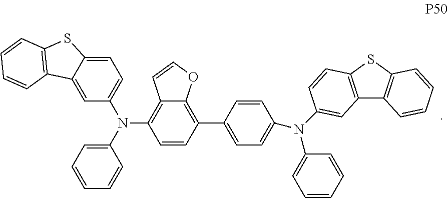 [SMILES: c1ccc(N(c2ccc(-c3ccc(N(c4ccccc4)c4ccc5sc6ccccc6c5c4)c4ccoc34)cc2)c2ccc3sc4ccccc4c3c2)cc1]

16. The benzo heterocyclic compound according to claim 15, wherein for a visible light having a wavelength in a range of 450 nm to 630 nm, the benzo heterocyclic compound has a refractive index of 1.85-2.50 and a light extinction coefficient less than 0.1; a difference between refractive indexes of the benzo heterocyclic compound for a visible light of 450 nm and a visible light of 550 nm is less than 0.35; and a difference between refractive indexes of the benzo heterocyclic compound for a visible light of 550 nm and a visible light of 630 nm is less than 0.15.

17. A display panel, comprising an organic light-emitting component, wherein the organic light-emitting component comprises an anode, a cathode, a light-emitting layer positioned between the anode and the cathode, and a capping layer covering the cathode, wherein the capping layer comprises one or more benzo heterocyclic compounds having a structure represented by Formula (I): 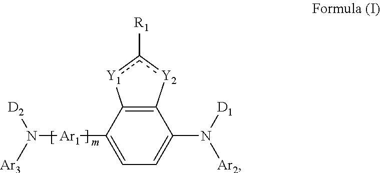
wherein R₁ is selected from a group consisting of hydrogen, C1-C20 alkyl, C1-C20 alkoxy, phenyl, naphthyl, anthracyl, and phenanthryl;
one of Y₁ and Y₂ is carbon, and the other one of Y₁ and Y₂ is oxygen or sulfur;
Ar₁ is selected from a group consisting of a substituted or unsubstituted phenyl, a substituted or unsubstituted naphthyl, a substituted or unsubstituted anthracyl, a substituted or unsubstituted phenanthryl, a substituted or unsubstituted acenaphthylenyl, and a substituted or unsubstituted aromatic heterocyclic group; m is 0 or 1;
D₁ and D₂ are each independently selected from a group consisting of a substituted or unsubstituted phenyl, a substituted or unsubstituted biphenyl, a substituted or unsubstituted naphthyl, a substituted or unsubstituted anthracyl, a substituted or unsubstituted phenanthryl, a substituted or unsubstituted acenaphthylenyl, a substituted or unsubstituted pyrenyl, a substituted or unsubstituted perylenyl, a substituted or unsubstituted fluorenyl, a substituted or unsubstituted spirodifluorenyl, a substituted or unsubstituted chrysenyl, a substituted or unsubstituted benzophenanthryl, a substituted or unsubstituted benzoanthracyl, a substituted or unsubstituted fluoranthenyl, a substituted or unsubstituted picenyl, a substituted or unsubstituted furyl, a substituted or unsubstituted benzofuryl, a substituted or unsubstituted dibenzofuryl, a substituted or unsubstituted thienyl, a substituted or unsubstituted benzothienyl, a substituted or unsubstituted dibenzothienyl, a substituted or unsubstituted phenoxazinyl, a substituted or unsubstituted phenazinyl, a substituted or unsubstituted phenothiazinyl, a substituted or unsubstituted thioxazinyl, a substituted or unsubstituted thianthrenyl, a carbazole-derived group, an acridine-derived group, diarylamino, a diarylamine-derived group, triarylamino and a triarylamine-derived group.

18. The display panel according to claim 17, wherein the capping layer has a thickness of 30 nm-100 nm, and
a transmittance of the cathode together with the capping layer for a visible light of 450 nm to 630 nm is greater than 65%.

19. The display panel according to claim 17, wherein the organic light-emitting component further comprises one or more of a hole injection layer, a hole transmission layer, an electron blocking layer, a hole blocking layer, an electron transmission layer, and an electron injection layer,
when the organic light-emitting component comprises the hole transmission layer, the hole transmission layer comprises one or more benzo heterocyclic compounds according to claim 1, and/or
when the organic light-emitting component comprises the light-emitting layer, the light-emitting layer comprises one or more benzo heterocyclic compounds according to claim 1.

20. The display panel according to claim 17, wherein the organic light-emitting component further comprises an auxiliary hole transmission layer positioned between the light-emitting layer and a hole transmission layer, and
the auxiliary hole transmission layer is configured to control a resonant period of light emitted from the light-emitting layer.

21. A display device, comprising a display panel comprising an organic light-emitting component, wherein the organic light-emitting component comprises an anode, a cathode, a light-emitting layer positioned between the anode and the cathode, and a capping layer covering the cathode, wherein the capping layer comprises one or more benzo heterocyclic compounds having a structure represented by Formula (I): 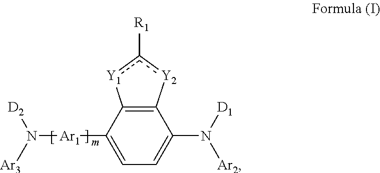
wherein R₁ is selected from a group consisting of hydrogen, C1-C20 alkyl, C1-C20 alkoxy, phenyl, naphthyl, anthracyl, and phenanthryl;
one of Y₁ and Y₂ is carbon, and the other one of Y₁ and Y₂ is oxygen or sulfur;
Ar₁ is selected from a group consisting of a substituted or unsubstituted phenyl, a substituted or unsubstituted naphthyl, a substituted or unsubstituted anthracyl, a substituted or unsubstituted phenanthryl, a substituted or unsubstituted acenaphthylenyl, and a substituted or unsubstituted aromatic heterocyclic group; m is 0 or 1;
D₁ and D₂ are each independently selected from a group consisting of a substituted or unsubstituted phenyl, a substituted or unsubstituted biphenyl, a substituted or unsubstituted naphthyl, a substituted or unsubstituted anthracyl, a substituted or unsubstituted phenanthryl, a substituted or unsubstituted acenaphthylenyl, a substituted or unsubstituted pyrenyl, a substituted or unsubstituted perylenyl, a substituted or unsubstituted fluorenyl, a substituted or unsubstituted spirodifluorenyl, a substituted or unsubstituted chrysenyl, a substituted or unsubstituted benzophenanthryl, a substituted or unsubstituted benzoanthracyl, a substituted or unsubstituted fluoranthenyl, a substituted or unsubstituted picenyl, a substituted or unsubstituted furyl, a substituted or unsubstituted benzofuryl, a substituted or unsubstituted dibenzofuryl, a substituted or unsubstituted thienyl, a substituted or unsubstituted benzothienyl, a substituted or unsubstituted dibenzothienyl, a substituted or unsubstituted phenoxazinyl, a substituted or unsubstituted phenazinyl, a substituted or unsubstituted phenothiazinyl, a substituted or unsubstituted thioxazinyl, a substituted or unsubstituted thianthrenyl, a carbazole-derived group, an acridine-derived group, diarylamino, a diarylamine-derived group, triarylamino and a triarylamine-derived group.
